(19) United States
(12) Patent Application Publication
(10) Pub. No.: US 2009/0012056 A1
(43) Pub. Date: Jan. 8, 2009

(54) Quinoline Compounds Capable of Binding the Cb2 and/or the 5-Ht6 Receptor

(75) Inventors: Mahmood Ahmed, Essex (GB); Gerard Martin Paul Giblin, Essex (GB); Christopher Norbert Johnson, Essex (GB); David George Hubert Livermore, Essex (GB); Neil Derek Miller, Essex (GB); Stephen Frederick Moss, Essex (GB); David R. Witty, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, Greenford, Middlesex (GB)

(21) Appl. No.: 12/088,502

(22) PCT Filed: Sep. 26, 2006
(86) PCT No.: PCT/EP2006/009416

§ 371 Date: Mar. 28, 2008

(30) Foreign Application Priority Data
Sep. 28, 2005 (GB) 0519765.2

(51) Int. Cl. A61K 31/4709; C07D 401/10; A61P 25/24

(52) U.S. Cl. 514/210.21; 546/153; 514/312

(57) ABSTRACT

The present invention relates to novel quinoline derivatives such as compounds of the formula (I) which possess antagonist potency for the 5-HT₆ receptor and/or are capable of selectively modulating the cannabinoid 2 receptor:

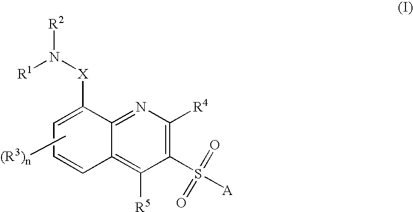

and the use of such compounds or pharmaceutical compositions thereof in the treatment of CNS disorders.

This invention relates to novel quinoline compounds having pharmacological activity, processes for their preparation, compositions containing them and their use in the treatment of diseases.

The quinoline compounds of the present invention may be useful in the treatment of diseases caused directly or indirectly by an increase or decrease in receptor binding at the cannabinoid receptor and/or the 5-HT₆ receptor. As such, the quinoline compounds of the present invention may be useful in the treatment of CNS disorders, in particular pain, Alzheimer's disease and age related cognitive decline.

Cannabinoids are a specific class of psychoactive compounds present in Indian cannabis (Cannabis sativa), including about sixty different molecules, the most representative being cannabinol, cannabidiol and several isomers of tetrahydrocannabinol. In addition to their well known psychoactive effects, over the years cannabinoids have also been used to alleviate pain.

The pathogenic mechanisms that give rise to pain symptoms can be grouped into two main categories:
those that are components of inflammatory tissue responses (Inflammatory Pain);
those that result from a neuronal lesion of some form (Neuropathic Pain).

With the advent of molecular biological techniques, the first cannabinoid receptor was found to be located mainly in the brain, in neural cell lines, and, only to a lesser extent, at the peripheral level. In view of its location, it was called the central receptor (“CB1”). See Matsuda et al., “Structure of a Cannabinoid Receptor and Functional Expression of the Cloned cDNA,” Nature, Vol. 346, pp. 561-564 (1990). The second cannabinoid receptor (“CB2”) was identified in the spleen, and was assumed to modulate the non psychoactive effects of the cannabinoids. See Munro et al., “Molecular Characterization of a Peripheral Receptor for Cannabinoids,” Nature, Vol. 365, pp. 61-65 (1993).

More recent data also suggests a role for CB2 receptor activation in the CNS. The CB2 receptor was thought to be restricted to the periphery, however emerging data suggests inflammatory pain-mediated induction of CB2 receptor expression in rat spinal cord which coincides with the appearance of activated microglia (Zhang et. al., 2003). Furthermore, CB2 receptor agonists have been shown to reduce mechanically evoked responses and wind-up of wide dynamic range neurones in spinal cord dorsal horn in animal models of inflammatory pain (Zhang et. al., 2003, Eur J. Neurosci. 17: 2750-2754, Nackley et. al., 2004, J. Neurophys. 92: 3562-3574, Elmes et. al., 2004, Eur. J. Neurosci. 20: 2311-2320).

The role of CB2 in immunomodulation, inflammation, osteoporosis, cardiovascular, renal and other disease conditions is now being examined.

Based on the foregoing, there is particular interest in compounds which have activity against the CB2 receptor and such compounds are believed to offer a unique approach toward the pharmacotherapy of pain (both inflammatory and neuropathic) immune disorders, inflammation, osteoporosis, renal ischemia and other pathophysiological conditions.

In light of the fact that cannabinoids act on receptors capable of modulating different functional effects, and in view of the low homology between CB2 and CB1 receptors, a class of drugs selective for the CB2 receptor sub-type is desirable. The natural or synthetic cannabinoids currently available do not fulfil this function because they are active at both CB2 receptors.

Another receptor associated with the CNS is the 5-HT₆ receptor. Messenger RNA that expresses this receptor is predominantly found in the brain (Raut et al., 1993, BioChem. Biophys. Res. Comms. 193: 268-276) and a number of CNS drugs are known to interact with the 5-HT₆ receptor (Monsma Jr. et al., 1993, Mol. Pharmacol. 43: 320-327). There is thus a considerable interest in identifying further compounds that bind to this receptor and their potential use in the treatment of CNS disorders.

Several classes of agonists and antagonists of the 5-HT₆ receptor have been disclosed in recent years (Glennon et al., 2003, J. Med. Chem. 46: 2795-2812) and evidence has emerged that antagonists of the 5-HT₆ receptor have a beneficial effect on memory consolidation in animal models of cognitive enhancement (Rogers et al., 2001, Psychopharmacology 158: 114-119, King et al., 2004, Neuropharmacology 47: 195-204). Consequently, the use of antagonists of the 5-HT₆ receptor has been suggested for the treatment of learning and memory disorders (Reavill et al., 2001, Curr. Op. Invest. Drugs 2: 104-109). As such, antagonists of the 5-HT₆ receptor may be of benefit in the treatment or prophylaxis of a number of CNS disorders, particularly Alzheimer's disease and age related cognitive decline.

A structurally novel class of compounds has now been found which are capable of selectively modulating the CB2 receptor over the CB1 receptor and/or which possess antagonist potency at the 5-HT₆ receptor. Compounds capable of selectively modulating the CB2 receptor may be antagonists, partial or full agonists, or inverse agonists. Compounds which possess antagonist potency at the 5-HT₆ receptor are capable of interfering with the physiological effects of 5-HT at the 5-HT₆ receptor and may be antagonists or inverse agonists.

The present invention therefore provides, in a first aspect, a compound of formula (I):

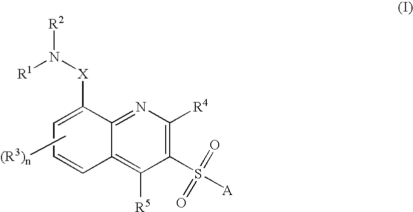

wherein:
R¹ and R² independently represent H, C₁₋₆ alkyl, or R¹ and R² together with the nitrogen atom to which they are attached form an optionally substituted 4 to 7 membered monocyclic heterocyclyl, a 9 to 11 membered bicyclic heterocyclyl, or a 10 membered spiro bicyclic heterocyclyl, any of which can optionally contain 1 or 2 further heteroatoms selected from O, N and S.
R³ represents halogen, —CN, —CF₃, —OCF₃, —OCHF₂, C₁₋₃ alkyl, C₁₋₃ alkoxy, —COC₁₋₃ alkyl, —NR⁶R⁷ or a group —CONR⁶R⁷;
R⁴ and R⁵ independently represent H, halogen, —CN, —CF₃, —OCF₃, —OCHF₂, C₁₋₃ alkyl, C₁₋₃ alkoxy, —COC₁₋₃ alkyl, —NR⁶R⁷ or a group —CONR⁶R⁷;
R⁶ and R⁷ independently represent H or C₁₋₃ alkyl;
X represents —(CH₂)ₘ— or —(CR⁸R⁹)—;
R⁸ and R⁹ independently represent H or C₁₋₃ alkyl;
m represents 2 to 4;
n represents 0 to 3; and
A represents an optionally substituted 6 to 10 membered aryl, an optionally substituted 5 to 7 membered monocyclic heteroaryl containing 1 to 3 heteroatoms selected from O, N and S, or a 9 to 10 membered fused bicyclic heteroaryl containing 1 to 3 heteroatoms selected from O, N and S;
or a pharmaceutically acceptable salt thereof.

When R¹ and R² together with the nitrogen atom to which they are attached form an optionally substituted 4 to 7 membered monocyclic heterocyclyl, a 9 to 11 membered bicyclic heterocyclyl, or a 10 membered spiro bicyclic heterocyclyl, the monocyclic, bicyclic or spiro bicyclic heterocyclyl may be substituted by one or more substituents (for example 1, 2 or 3), which may be the same or different, selected from the group consisting of halogen, oxo, hydroxyl, —CN, nitro, —NR⁶R⁷, —CONR⁶R⁷, —CF₃, trifluoroethyl, —OCF₃, —OCHF₂, C₁₋₄ alkyl, C₁₋₄ alkoxy, C₁₋₄ alkylthio, —COC₁₋₄ alkyl and C₁₋₄ alkylsulfonyl. In one embodiment, the optional substituents of the monocyclic or bicyclic heterocyclyl are selected from the group consisting of halogen, oxygen, C₁₋₄ alkyl, C₁₋₄ alkoxy and —COC₁₋₄ alkyl.

When A is an 5 to 7 membered monocyclic heteroaryl or a 9 to 10 membered fused bicyclic heteroaryl it may be substituted by one or more substituents (for example 1, 2 or 3), which may be the same or different, selected from the group consisting of halogen, hydroxyl, —CN, nitro, —NR⁶R⁷, —CONR⁶R⁷, —CF₃, —OCF₃, —OCHF₂, C₁₋₆ alkyl, C₁₋₆ alkoxy, —COC₁₋₆ alkyl, —COC₁₋₆ alkoxy, —NHCOC₁₋₆ alkyl and —COOH. In one embodiment, the optional substituents of the aryl or heteroaryl are selected from the group consisting of halogen, C₁₋₃ alkyl, C₁₋₃ alkoxy and —NHCOC₁₋₃ alkyl.

As used herein, the term “alkyl” (when used as a group or as part of a group) refers to a straight or branched hydrocarbon chain containing the specified number of carbon atoms. For example, C₁₋₆ alkyl means a straight or branched hydrocarbon chain containing at least 1 and at most 6 carbon atoms. Examples of alkyl include, but are not limited to; methyl (Me), ethyl (Et), n-propyl, i-propyl, n-hexyl and i-hexyl.

As used herein, the term “alkoxy” (when used as a group or as part of a group) refers to an alkyl ether radical, wherein the term “alkyl” is defined above. Examples of alkoxy include, but are not limited to; methoxy, ethoxy, n-propoxy, i-propoxy, n-pentoxy and i-pentoxy.

The term ‘halogen’ is used herein to describe a group selected from fluorine, chlorine, bromine and iodine.

The term ‘aryl’ as used herein refers to a C₆₋₁₀ monocyclic or bicyclic hydrocarbon ring wherein at least one ring is aromatic. Examples of such groups include phenyl and naphthyl.

The term “heteroaryl”, unless stated otherwise, is intended to mean a 5 to 7 membered monocyclic aromatic or a fused 9 to 10 membered bicyclic aromatic ring containing 1 to 3 heteroatoms selected from oxygen, nitrogen and sulfur. Suitable examples of such monocyclic aromatic rings include thienyl, furanyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused bicyclic aromatic rings include quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom except where indicated otherwise.

It will be appreciated that wherein the above mentioned aryl or heteroaryl groups have more than one substituent, said substituents may be linked to form a ring.

The term “heterocyclyl” is intended to mean a 4-7 membered monocyclic saturated or partially unsaturated aliphatic ring containing 1 to 3 heteroatoms selected from oxygen, nitrogen or sulphur (referred to as a monocyclic heterocyclyl); or a 5-7 membered monocyclic saturated or partially unsaturated aliphatic ring containing 1 to 3 heteroatoms selected from oxygen, nitrogen or sulphur fused to a benzene or monocyclic heteroaryl ring (referred to as a bicyclic heterocyclyl); or a 10 membered saturated or partially unsaturated aliphatic bicyclic ring system containing 1 to 3 heteroatoms selected from oxygen, nitrogen or sulphur, wherein the two rings share a single carbon atom (referred to as a spiro bicyclic heterocyclyl). Suitable examples of such monocyclic heterocyclyls include pyrrolidinyl, piperidinyl, piperazinyl, morpholinyl, thiamorpholinyl, diazepanyl, azepanyl, dihydroimidazolyl, tetrahydropyranyl, tetrahydrothiapyranyl and tetrahydrofuranyl. Suitable examples of such bicyclic heterocyclyls include dihydroindolyl, dihydroisoindolyl, tetrahydroquinolinyl, tetrahydrobenzazepinyl and tetrahydroisoquinolinyl. A suitable example of such a spiro bicyclic heterocyclyl is 1,4-dioxa-8-azaspiro[4.5]decane.

In certain embodiments, R¹ and R² independently represent H, C₁₋₆alkyl, or R¹ and R² together with the nitrogen atom to which they are attached form an optionally substituted 4 to 7 membered monocyclic heterocyclyl selected from the group consisting of azetidinyl, pyrrolidinyl, piperidinyl, piperazinyl, morpholinyl and thiomorpholinyl, or form an optionally substituted 1,4-dioxa-8-azaspiro[4.5]decane spiro bicyclic heterocyclyl. In one embodiment, the optional substituent of the 4 to 7 membered monocyclic heterocyclyl is fluorine and one of the carbon atoms of the monocyclic heterocyclyl is disubstituted with 2 fluorine atoms.

In one embodiment, m represents 2 or 3.

In one embodiment, X represents —CH₂—.

In certain embodiments, R³ represents halogen, —CN, or C₁₋₃ alkyl. In one embodiment, R³ represents Cl or methyl.

In one embodiment, n represents 0.

In certain embodiments, R⁴ and R⁵ independently represent H, halogen or methyl. In one embodiment, R⁴ and R⁵ both represent hydrogen.

In one particular embodiment, n represents 0 and R⁴ and R⁵ both represent hydrogen.

In one embodiment, R⁶ and R⁷ independently represent hydrogen or methyl.

In one embodiment, R⁸ and R⁹ independently represent hydrogen or methyl.

In certain embodiments, A represents an optionally substituted phenyl or naphthyl.

In certain embodiments, there is provided a compound of formula (I) wherein: R¹ and R² independently represent H, C₁₋₆ alkyl, or R¹ and R² together with the nitrogen atom to which they are attached form an optionally substituted 4 to 7 membered monocyclic heterocyclyl selected from the group consisting of azetidinyl, pyrrolidinyl, piperidinyl, piperazinyl, morpholinyl and thiomorpholinyl, or form an optionally substituted 1,4-dioxa-8-azaspiro[4.5]decane spiro bicyclic heterocyclyl, and wherein the optional substituents of the monocyclic heterocyclyl or the spiro bicyclic heterocyclyl are selected from the group consisting of fluorine, methyl, methoxy and COCH₃;

X represents —(CH₂)ₘ— or —(CR⁸R⁹)—;
m represents 2 or 3;
R⁸ and R⁹ independently represent H or methyl;
R³ represents halogen or C₁₋₃ alkyl;
n represents 0 to 3;
R⁴ and R⁵ independently represent H, halogen or methyl; and
A represents optionally substituted phenyl or naphthyl, wherein the optional substituents are selected from the group consisting of halogen, C₁₋₃ alkyl, C₁₋₃ alkoxy and —NHCOC₁₋₃ alkyl;
or a pharmaceutically acceptable salt thereof.

Particular compounds according to the invention include examples E1-E46 as shown below, or a pharmaceutically acceptable salt thereof.

The compounds of formula (I) can form acid addition salts thereof. It will be appreciated that for use in medicine the salts of the compounds of formula (I) should be pharmaceutically acceptable. Pharmaceutically acceptable salts include those described by Berge, Bighley and Monkhouse, J. Pharm. Sci., 1977, 66, 1-19. The term “pharmaceutically acceptable salts” includes salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, trishydroxylmethyl amino methane, tripropyl amine, tromethamine, and the like. When a compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Examples of pharmaceutically acceptable salts include the ammonium, calcium, magnesium, potassium, and sodium salts, and those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The subject invention also includes isotopically-labeled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as 3H, 11C, 14C, 18F, 123I and 125I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as 3H, 14C are incorporated, are useful in drug and/or substrate tissue distribution assays. Tritiated, i.e., 3H, and carbon-14, i.e., 14C, isotopes are particularly preferred for their ease of preparation and detectability. 11C and 8F isotopes are particularly useful in PET (positron emission tomography), and 125I isotopes are particularly useful in SPECT (single photon emission computerized tomography), all useful in brain imaging. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labeled compounds of formula (I) and following of this invention can generally be prepared by carrying out the procedures disclosed in the Schemes and/or in the Examples below, by substituting a readily available isotopically labeled reagent for a non-isotopically labeled reagent.

The present invention also provides a process for the preparation of a compound of formula (I) or a pharmaceutically acceptable salt, which process comprises:

(a) reacting a compound of formula (II)

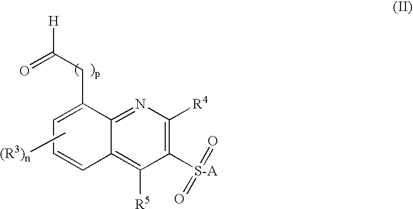

or an optionally protected derivative thereof, wherein R³, R⁴, R⁵, n and A are as defined above and p represents 0 to 3, with a compound of formula HNR¹R² wherein R¹ and R² are as defined above, and optionally thereafter removing any protecting groups; or

(b) reacting a compound of formula (III)

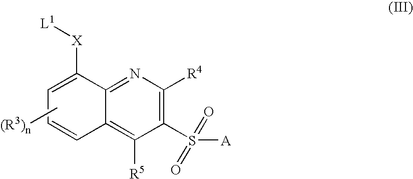

or an optionally protected derivative thereof; wherein R³, R⁴, R⁵, X, n and A are as defined above and L¹ represents a leaving group such as a halogen atom or an alkylsulfonyloxy or arylsulfonyloxy group (e.g. methylsulfonyloxy), with a compound of formula HNR¹R² as defined above, and optionally thereafter removing any protecting groups;

(c) deprotecting a compound of formula (I) which is protected;

(d) interconversion to other compounds of formula (I) and/or forming a pharmaceutically acceptable salt and/or solvate.

Process (a) typically comprises the use of a reducing agent such as sodium cyanoborohydride or sodium triacetoxyborohydride in a suitable solvent such as ethanol, dichloromethane or 1,2-dichloroethane.

Process (b) is typically carried out in the presence of a base such as triethylamine or an excess of the compound of formula HNR¹R² in a suitable solvent such as a C₁₋₆ alcohol (e.g. isopropanol), optionally at elevated temperature (e.g. under reflux conditions).

In processes (a), (b) and (c), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid. A further amine protecting group includes methyl which may be removed using standard methods for N-dealkylation (e.g. 1-chloroethyl chloroformate under basic conditions followed by treatment with methanol).

Process (d) may be performed using conventional interconversion procedures such as epimerisation, oxidation, reduction, reductive alkylation, alkylation, nucleophilic or electrophilic aromatic substitution, ester hydrolysis or amide bond formation. For example, N-dealkylation of a compound of formula (I) wherein R¹ or R² represents an alkyl group to give a compound of formula (I) wherein R¹ or R² represents hydrogen. It will be appreciated that such interconversion may be interconversion of protected derivatives of formula (I) which may subsequently be deprotected following interconversion.

In addition, process (d) may also comprise, for example, reacting a compound of formula (I), wherein R¹ or R² represents hydrogen, with an aldehyde or ketone in the presence of a reducing agent in order to generate a compound of formula (I) where R¹ or R² represents C₁₋₆alkyl. This may be performed using a hydride donor agent such as sodium cyanoborohydride, sodium triacetoxyborohydride or a resin bound form of cyanoborohydride in an alcoholic solvent such as ethanol and in the presence of an acid such as acetic acid, or under conditions of catalytic hydrogenation. Alternatively, such a transformation may be carried out by reacting a compound of formula (I), wherein R¹ or R² represents hydrogen, with a compound of formula R^{1a}-L or R^{2a}-L, wherein R^{1a} and R^{2a} represent C₁₋₆alkyl and L represents a leaving group such as a halogen atom (e.g. bromine or iodine) or methylsulfonyloxy group, optionally in the presence of a suitable base such as potassium carbonate or triethylamine using an appropriate solvent such as N,N-dimethylformamide or a C₁₋₄alkanol.

Compounds of formula (II) may be prepared by oxidative cleavage of a compound of formula (IV)

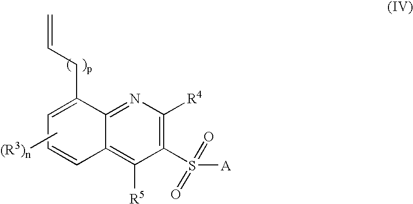

wherein R³, R⁴, R⁵, n and A are as defined above and p represents 0 to 3. Such a process may be effected using ozone in a suitable solvent such as dichloromethane in the presence of a C₁₋₄ alkanol (e.g. methanol), followed by reductive work-up, for example using thiourea or dimethyl sulfide, then hydrolysis of the intermediate acetal or ketal under acid conditions, for example using a dilute aqueous mineral acid such as hydrochloric or sulfuric acid, or an organic acid such as trifluoroacetic acid in a suitable solvent such as dichloromethane.

Compounds of formula (IV) may be prepared according to the following process:

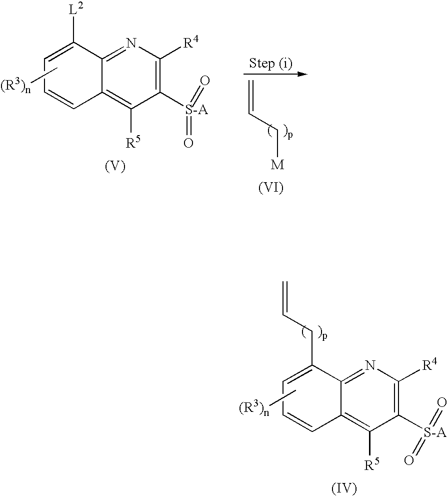

wherein R³, R⁴, R⁵, n and A are as defined above and p represents 0 to 3, L² represents a leaving group such as a bromine or iodine atom or a trifluoromethylsulfonyloxy group and M is a metal residue such as trialkylstannyl, e.g. tributylstannyl.

Step (i) typically comprises the use of palladium such as palladium (II) acetate and a ligand such as tris-(2-furyl)phosphine using an appropriate solvent such as 1,4-dioxane.

Compounds of formula (II) wherein p represents 3 may be prepared by reaction of compounds of formula (V) as defined above with compounds of formula (VII)

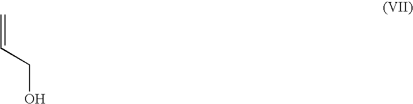

in the presence of a palladium catalyst such as palladium (II) acetate, a base such as sodium hydrogen carbonate and an additive such as tetrabutylammonium chloride in a suitable solvent such as N,N-dimethylformamide.

Compounds of formula (III) may be prepared by reduction of compounds of formula (II) as defined above using a suitable reducing agent such as sodium borohydride, then conversion of the resulting alcohol to leaving group L using standard methodology, for example using methylsulfonyl chloride in the presence of a suitable base such as pyridine in an appropriate solvent.

Compounds of formula (V) as defined above are described in WO 03/080580.

Compounds of formula (VI) and (VII) as defined above are known in the literature or may be prepared using analogous methods.

Compounds of formula (I) wherein X represents —(R⁸R⁹)—, R⁸ represents C₁₋₃ alkyl and R⁹ represents hydrogen may be prepared according to the following process:

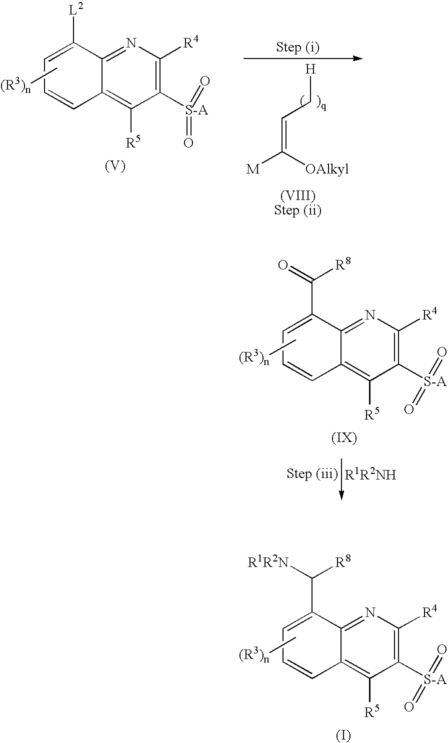

Step (i) typically comprises the coupling of a compound of formula (V) wherein R³, R⁴, R⁵, n and A are as defined above and L² represents a leaving group such as a bromine or iodine atom or a trifluoromethylsulfonyloxy group, with a metallovinyl compound of formula (VIII) where q represents 0 to 2 and M is a metal residue such as trialkylstannyl, e.g. tributylstannyl using a palladium catalyst such as dichloro bis(triphenylphosphine) palladium (II) in an appropriate solvent such as toluene.

Step (ii) is the hydrolysis of the first formed vinyl ether using, for example, an aqueous mineral acid such as hydrochloride acid or an aqueous organic acid such as trifluoroacetic acid or formic acid.

Step (iii) comprises the reductive amination of compound (IX) with an amine of formula R¹R²NH wherein R¹ and R² are as defined above. This may be typically be performed using a hydride donor agent such as sodium cyanoborohydride, sodium triacetoxyborohydride or a resin bound form of cyanoborohydride in suitable solvent such as dichloromethane, 1,2-dichloroethane or ethanol and in the presence of an acid such as hydrochloric acid or acetic acid, or under conditions of catalytic hydrogenation.

Compounds of formula (I) wherein X represents —(R⁸R⁹)— and R⁸ and R⁹ represent C₁₋₃ alkyl may be prepared according to the following process:

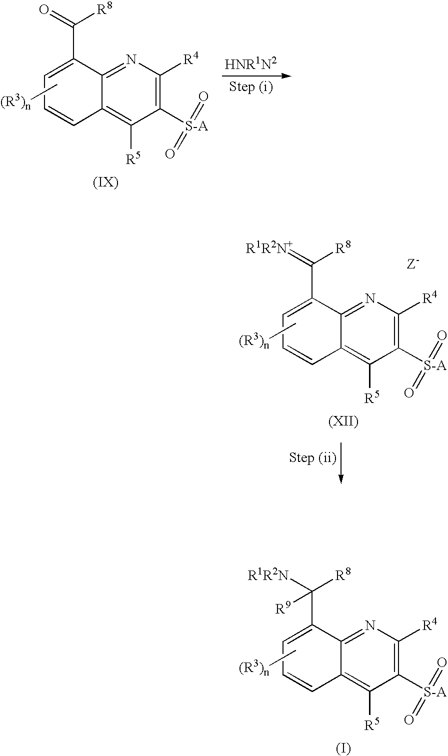

Step (i) comprises the formation of an iminium salt (XII) where Z=the anion, by the condensation of a ketone of formula (IX) where R³, R⁴, R⁵, n and A are as defined above and R⁸═C₁₋₃ alkyl, with the salt, for example a tetrafluoroborate salt, of a secondary amine of formula NHR¹R² where R¹ and R² are as defined above in the presence of a dehydrating agent such as molecular sieves, or by azeotropic removal of water under Dean and Stark conditions in a suitable solvent such as toluene.

Step (ii) entails the alylkation of the iminium salt (XII) by reaction with a organometrallic agent such as a Grignard reagent, where R⁹═C₁₋₃ alkyl, (for example methyl magnesium bromide R⁹=Me), typically in the presence of a cerium salt such as cerium chloride, in a suitable solvent, such as tetrahydrofuran.

Compounds of formula (I) may also be prepared according to the following process:

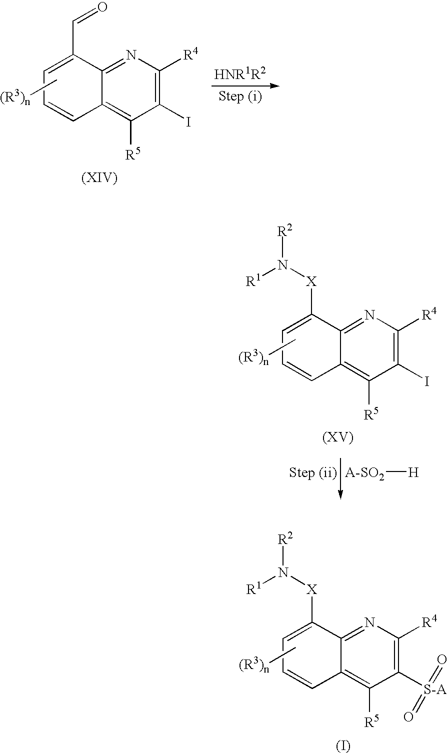

wherein R¹, R², R³, R⁴, R⁵, n and A are as defined above.

Step (i) comprises dissolving a compound of formula (XIV) and a compound of formula HNR¹R² in an appropriate solvent, for example dichloromethane, adding sodiumtriacetoxyborohydride and acetic acid, and then leaving the reaction to progress under an inert atmosphere, for example argon.

Step (ii) comprises reacting a compound of formula (XV) with a compound of formula A-SO₂—H or a suitable salt thereof in the presence of a base, for example potassium carbonate, a metal catalyst, for example copper (I) iodide, and a diamine ligand, for example N,N-dimethylethylenediamine, using an appropriate solvent such as dimethylsulfoxide.

Pharmaceutically acceptable salts may be prepared conventionally by reaction with the appropriate acid or acid derivative.

Compounds of the invention may bind to the CB2 receptor with greater affinity that to the CB1 receptor; such compounds may be particularly useful in treating CB2 receptor mediated diseases. In one embodiment compounds of formula (I) have an EC50 value at the cloned human cannabinoid CB2 receptor of at least 50 times the EC50 values at the cloned human cannabinoid CB1 receptor and/or have less than 30% efficacy at the CB1 receptor.

It is believed that compounds of the invention which bind to the CB2 receptor may be useful in the treatment of the disorders that follow. Thus, compounds of formula (I) may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome (IBS); pain associated with myocardial ischemia; post operative pain; headache; toothache; and dysmenorrhea.

Compounds of the invention which bind to the CB2 receptor may also have disease modification or joint structure preservation properties in multiple sclerosis, rheumatoid arthritis, osteo-arthritis, rheumatoid spondylitis, gouty arthritis and juvenile arthritis.

Compounds of the invention which bind to the CB2 receptor may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of formula (I) which bind to the CB2 receptor may also be useful in the treatment of fever.

Compounds of formula (I) which bind to the CB2 receptor may also be useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastroesophageal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, tendinitis, bursitis, and Sjogren's syndrome.

Compounds of formula (I) which bind to the CB2 receptor may also be useful in the treatment of bladder hyperrelexia following bladder inflammation.

Compounds of formula (I) which bind to the CB2 receptor may also be useful in the treatment of immunological diseases such as autoimmune diseases, immunological deficiency diseases or organ transplantation.

The compounds of formula (I) which bind to the CB2 receptor may also be effective in increasing the latency of HIV infection.

Compounds of formula (I) which bind to the CB2 receptor may also be useful in the treatment of diseases of abnormal platelet function (e.g. occlusive vascular diseases).

Compounds of formula (I) which bind to the CB2 receptor may also be useful in the treatment of neuritis, heart burn, dysphagia, pelvic hypersensitivity, urinary incontinence, cystitis or pruritis.

Compounds of formula (I) which bind to the CB2 receptor may also be useful for the preparation of a drug with diuretic action.

Compounds of formula (I) which bind to the CB2 receptor may also be useful in the treatment of impotence or erectile dysfunction.

Compounds of formula (I) which bind to the CB2 receptor may also be useful for attenuating the hemodynamic side effects of non-steroidal anti-inflammatory drugs (NSAID's) and cyclooxygenase-2 (COX-2) inhibitors.

Compounds of formula (I) which bind to the CB2 receptor may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflamation.

Compounds of formula (I) which bind to the CB2 receptor may also be useful in neuroprotection and in the treatment of neurodegeneration following stroke, cardiac arrest, pulmonary bypass, traumatic brain injury, spinal cord injury or the like.

Compounds of formula (I) which bind to the CB2 receptor may also be useful in the treatment of tinnitus.

Compounds of formula (I) which bind to the CB2 receptor may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof.

Compounds of formula (I) which bind to the CB2 receptor may also be useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

Compounds of formula (I) which bind to the CB2 receptor may also be useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

Compounds of formula (I) which bind to the 5-HT₆ receptor may be useful in the treatment of certain CNS disorders such as anxiety, depression, epilepsy, obsessive compulsive disorders, migraine, cognitive memory disorders (e.g. Alzheimers disease, age related cognitive decline, mild cognitive impairment and vascular dementia), Parkinsons Disease, ADHD (Attention Deficit Disorder/Hyperactivity Syndrome), sleep disorders (including disturbances of Circadian rhythm), feeding disorders such as anorexia and bulimia, panic attacks, withdrawal from drug abuse such as cocaine, ethanol, nicotine and benzodiazepines, schizophrenia (in particular cognitive deficits of schizophrenia), stroke and also disorders associated with spinal trauma and/or head injury such as hydrocephalus.

Compounds of the invention which bind to the 5-HT₆ receptor may also be useful in the treatment of certain GI (gastrointestinal) disorders such as IBS.

Compounds of the invention which bind to the 5-HT₆ receptor may also be useful in the treatment of obesity.

Compounds of the invention which bind to both the CB2 and the 5-HT₆ receptor may be particularly useful in the treatment of certain CNS disorders such as anxiety, depression, obsessive compulsive disorders, cognitive disorders (e.g. Alzheimer's disease, age related cognitive decline and mild cognitive impairment), Parkinson's Disease, sleep disorders, feeding disorders such as anorexia and bulimia, panic attacks, withdrawal from drug abuse such as cocaine, ethanol, nicotine and benzodiazepines, schizophrenia (in particular cognitive deficits of schizophrenia), stroke and also disorders associated with spinal trauma and/or head injury.

Compounds of the invention which bind to both the CB2 and the 5-HT₆ receptor may also be particularly useful in the treatment of IBS.

The term “treatment” or “treating” as used herein includes the treatment of established disorders and also includes the prophylaxis thereof. The term “prophylaxis” is used herein to mean preventing symptoms in an already afflicted subject or preventing recurrence of symptoms in an afflicted subject and is not limited to complete prevention of an affliction.

According to a further aspect of the invention, we provide a compound of formula (I) which binds to the CB2 and/or the 5-HT₆ receptor, or a pharmaceutically acceptable salt thereof, for use in human or veterinary medicine.

According to another aspect of the invention, we provide a compound of formula (I) which binds to the CB2 receptor, or a pharmaceutically acceptable salt thereof, for use in the treatment of a condition which is mediated by the activity of cannabinoid 2 receptors.

According to a further aspect of the invention, we provide a method of treating a mammal, for example a human suffering from a condition which is mediated by the activity of the CB2 receptor which comprises administering to said subject a therapeutically effective amount of a compound of formula (I) which binds to the CB2 receptor or a pharmaceutically acceptable salt thereof.

According to a further aspect of the invention we provide a method of treating a mammal, for example a human suffering from an immune disorder, an inflammatory disorder, pain, rheumatoid arthritis, multiple sclerosis, osteoarthritis or osteoporosis which method comprises administering to said subject an effective amount of a compound of formula (I) which binds to the CB2 receptor or a pharmaceutically acceptable salt thereof.

In one embodiment the pain is selected from inflammatory pain, visceral pain, cancer pain, neuropathic pain, lower back pain, muscular skeletal, post operative pain, acute pain and migraine. For example, the inflammatory pain is pain associated with rheumatoid arthritis or osteoarthritis.

According to another aspect of the invention there is provided the use of a compound of formula (I) which binds to the CB2 receptor, or a pharmaceutically salt thereof, for the manufacture of a therapeutic agent for the treatment or prevention of a condition such as an immune disorder, an inflammatory disorder, pain, rheumatoid arthritis, multiple sclerosis, osteoarthritis or osteoporosis.

According to another aspect of the invention, we provide a compound of formula (I) which binds to the 5-HT₆ receptor, or a pharmaceutically acceptable salt thereof, for use in the treatment of a condition which is mediated by the activity of 5-HT₆ receptors.

According to a further aspect of the invention, we provide a method of treating a mammal, for example a human suffering from a condition which is mediated by the activity of 5-HT₆ receptors which comprises administering to said subject a therapeutically effective amount of a compound of formula (I) which binds to the 5-HT₆ receptor or a pharmaceutically acceptable salt thereof.

According to a further aspect of the invention we provide a method of treating a mammal, for example a human suffering from a CNS disorder, which method comprises administering to said subject an effective amount of a compound of formula (I) which binds to the 5-HT₆ receptor or a pharmaceutically acceptable salt thereof.

In one embodiment the CNS disorder is selected from anxiety, depression, obsessive compulsive disorders, cognitive disorders, Parkinson's Disease, sleep disorders, feeding disorders such as anorexia and bulimia, panic attacks, withdrawal from drug abuse such as cocaine, ethanol, nicotine and benzodiazepines, schizophrenia, stroke and also disorders associated with spinal trauma and/or head injury. For example, the cognitive disorders are disorders associated with Alzheimer's Disease, age related cognitive decline, mild cognitive impairment and vascular dementia.

According to another aspect of the invention there is provided the use of a compound of formula (I) which binds to the 5-HT₆ receptor, or a pharmaceutically acceptable salt thereof, for the manufacture of a therapeutic agent for the treatment or prevention of a condition such as cognitive disorders (for example, cognitive disorders associated with Alzheimer's Disease, age related cognitive decline, mild cognitive impairment and vascular dementia), Parkinson's Disease, schizophrenia, IBS or obesity.

According to a further aspect of the invention we provide a method of treating a mammal, for example a human suffering from certain CNS disorders such as anxiety, depression, obsessive compulsive disorders, cognitive disorders (e.g. Alzheimer's Disease, age related cognitive decline and mild cognitive impairment), Parkinson's Disease, sleep disorders, feeding disorders such as anorexia and bulimia, panic attacks, withdrawal from drug abuse such as cocaine, ethanol, nicotine and benzodiazepines, schizophrenia (in particular cognitive deficits of schizophrenia), stroke and also disorders associated with spinal trauma and/or head injury, which method comprises administering to said subject an effective amount of a compound of formula (I) which binds to the CB2 receptor and the 5-HT₆ receptor or a pharmaceutically acceptable salt thereof.

According to another aspect of the invention there is provided the use of a compound of formula (I) which binds to the CB2 receptor and the 5-HT₆ receptor, or a pharmaceutically acceptable salt thereof, for the manufacture of a therapeutic agent for the treatment or prevention of a condition such as Alzheimer's Disease, age related cognitive decline, mild cognitive impairment, schizophrenia or stroke.

In order to use a compound of formula (I) or a pharmaceutically acceptable salt thereof for the treatment of humans and other mammals it is normally formulated in accordance with standard pharmaceutical practice as a pharmaceutical composition. Therefore in another aspect of the invention is provided a pharmaceutical composition comprising a compound of formula (I), or a pharmaceutically acceptable salt thereof, adapted for use in human or veterinary medicine.

As used herein, the expression “compounds capable of selectively modulating the CB2 receptor” means both antagonists, partial or full agonists and inverse agonists. In one embodiment the present compounds capable of selectively modulating the CB2 receptor are agonists.

Compounds which possess antagonist potency at the 5-HT₆ receptor are capable of interfering with the physiological effects of 5-HT at the 5-HT₆ receptor and may be antagonists or inverse agonists. In one embodiment the present compounds capable of interfering with the physiological effects of 5-HT at the 5-HT₆ receptor are antagonists.

5-HT₆ antagonists have the potential to be capable of increasing basal and learning-induced polysialylated neuron cell frequency in brain regions such as the rat medial temporal lobe and associated hippocampus, as described in WO 03/066056. Thus, according to a further aspect of the present invention, we provide a method of promoting neuronal growth within the central nervous system of a mammal which comprises the step of administering a compound of formula (I) or a pharmaceutically acceptable salt thereof.

In order to use the compounds of formula (I) in therapy, they will normally be formulated into a pharmaceutical composition in accordance with standard pharmaceutical practice. The present invention also provides a pharmaceutical composition, which comprises a compound of formula (I), or a pharmaceutically acceptable salt thereof, and optionally a pharmaceutically acceptable carrier.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 200 mg, for example 20 to 40 mg; and such unit doses will preferably be administered once a day, although administration more than once a day may be required; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention but are not intended to be limiting.

### Description 1

### 8-Ethenyl-3-(phenylsulfonyl)quinoline

### D1

A stirred mixture of 8-iodo-3-phenylsulfonylquinoline (1 g, 2.5 mmol) (see WO03/080580 for preparation), vinyl tributyl stannane (0.74 ml, 2.5 mmol), palladium (II) acetate (10 mg, 0.045 mmol) and tri(2-furyl)phosphine (40 mg, 0.172 mmol) in 1,4-dioxan (20 ml) was heated at 90° C. for 3.5 h under argon. The reaction mixture was cooled to ambient temperature, diluted with dichloromethane (100 ml) and the mixture was washed with water (2×50 ml). The organic phase was dried (MgSO₄) and evaporated in vacuo to a solid which was stirred at ambient temperature with diethyl ether (30 ml) for 0.5 h. The material was filtered to give the title compound (D1) as a yellow solid (0.65 g, 2.2 mmol, 88%). Mass Spectrum: C₁₇H₁₃NO₂S requires 295; found 296 (MH⁺)

### Description 2

### 8-[Bis(methyloxy)methyl]-3-(phenylsulfonyl)quinoline

### D2

A solution of 8-ethenyl-3-(phenylsulfonyl)quinoline (D1) (0.48 g, 1.6 mmol) in dichloromethane (40 ml) was diluted with methanol (20 ml) and the stirred solution was cooled to −60° C. under argon. At this temperature, oxygen was passed through the solution using a Fischer Model 500 Ozone Generator at a flow rate of approx. 25 L/hour and a current of 0.4 A was applied to generate ozone gas. After 1 h under these conditions, the applied current was switched off and the flow of oxygen through the cooled solution (−60° C.) was maintained until the emerging gas showed a negative test with moist starch-iodide paper. The stirred solution was then purged with argon at −60° C. for 5 mins and dimethyl sulfide (0.77 ml, 10.5 mmol) was added. The stirred solution was allowed to warm to ambient temperature over 18 h. The solution was then diluted with dichloromethane (50 ml) and washed with water (2×50 ml), dried (MgSO₄) and evaporated in vacuo to a brown oil. This material was purified by column chromatography over silica gel using an ethyl acetate/pentane solvent gradient to afford the title compound (D2) as an oil (0.332 g, 1.0 mmol, 60%).

δH (CDCl₃, 250 MHz) 3.44 (6H, s), 6.50 (1H, s), 7.50-7.76 (4H, m), 7.92-8.15 (4H, m), 8.82 (1H, d, J=2 Hz), 9.30 (1H, d, J=2 Hz).

MS: No molecular ion found for C₁₈H₁₇NO₄S.

### Description 3

### 3-(Phenylsulfonyl)-8-quinolinecarbaldehyde

### D3

A solution of 8-[bis(methyloxy)methyl]-3-(phenylsulfonyl)quinoline (D2) (0.295 g, 0.86 mmol) in 1,2-dichloroethane (10 ml) and trifluoroacetic acid (0.1 ml, 1.3 mmol) was heated at 50° C. for 3.8 h. The solution was evaporated in vacuo to a solid which was identified as the title compound (D3) (0.243 g, 0.82 mmol, 95%).

Mass Spectrum: C₁₆H₁₁NO₃S requires 297; found 298 (MH⁺)

### Description 4

### 3-(Phenylsulfonyl)-8-(2-propen-1-yl)quinoline

### D4

A stirred mixture of 8-iodo-3-phenylsulfonylquinoline (1.15 g, 2.9 mmol), tributyl(2-propen-1-yl)stannane (1.36 ml, 4.4 mmol) and tetrakis(triphenylphosphine))palladium (0) (0.16 g, 0.14 mmol) in N,N-dimethylformamide (16 ml) was heated in a sealed reaction vessel at 150° C. for 10 mins in an Emrys Optimizer microwave apparatus. The cooled reaction mixture was filtered through a bed of celite under suction and the filtrate diluted with ethyl acetate (100 ml). This solution was washed with 5% brine (3×100 ml), dried (MgSO₄) and evaporated in vacuo to a brown oil which was purified by column chromatography over silica gel using an ethyl acetate/pentane solvent gradient to give a white solid. This solid was stirred with diethyl ether (10 ml), filtered and identified as the title compound (D4) (0.58 g, 1.9 mmol, 66%)

δH (CDCl₃, 250 MHz) 4.03 (2H, d, J=6.6 Hz), 5.06-5.14 (2H, m), 6.04-6.21 (1H, m), 7.53-7.64 (4H, m), 7.75 (1H, dd, J=2, 8 Hz), 7.85 (1H, dd, J=2, 8 Hz), 8.04-8.08 (2H, m), 8.80 (1H, d, J=2 Hz), 9.25 (1H, d, J=2 Hz).

Mass Spectrum: C₁₈H₁₅NO₂S requires 309; found 310 (MH⁺).

### Description 5

### 8-[2,2-Bis(methyloxy)ethyl]-3-(phenylsulfonyl)quinoline

### D5

The title compound (D5) was prepared by ozonolysis in 40% yield as described in Description 2, using 3-(phenylsulfonyl)-8-(2-propen-1-yl)quinoline (D4) (0.58 g, 1.9 mmol).

δH (CDCl₃, 250 MHz) 3.34 (6H, s), 3.55 (2H, d, J=5.6 Hz), 4.83 (1H, t, J=5.6 Hz), 7.52-7.65 (4H, m), 7.79-7.89 (2H, m), 8.04-8.08 (2H, m), 8.80 (1H, d, J=2 Hz), 9.29 (1H, d, J=2 Hz).

MS: No molecular ion found for C₁₉H₁₉NO₄S.

### Description 6

### [3-(Phenylsulfonyl)-8-quinolinyl]acetaldehyde

### D6

The title compound (D6) was prepared as described in Description 3, from 8-[2,2-bis(methyloxy)ethyl]-3-(phenylsulfonyl)quinoline (D5) (0.27 g, 0.76 mmol), 1,2-dichloroethane (10 ml) and trifluoroacetic acid (0.187 ml, 2.4 mmol).

δH (CDCl₃, 250 MHz) 4.24 (2H, d, J=1.9 Hz), 7.55-7.80 (5H, m), 7.92-8.05 (3H, m), 8.95 (1H, d, J=2 Hz), 9.39 (1H, d, J=2 Hz), 9.58 (1H, s).

Mass Spectrum: C₁₇H₁₃NO₃S requires 311; found 312 (MH⁺).

This material was used directly in the next stage (see Examples E7 and E8) without purification.

### Description 7

### 3-[3-(Phenylsulfonyl)-8-quinolinyl]propanal

### D7

Allyl alcohol (0.39 ml, 5.7 mmol) was added to a mixture of 8-iodo-3-phenylsulfonylquinoline (1.5 g, 3.8 mmol), palladium (II) acetate (17 mg, 0.076 mmol), anhydrous sodium hydrogen carbonate (0.8 g, 9.5 mmol) and anhydrous tetra-n-butylammonium chloride (1.06 g, 3.8 mmol) in de-gassed N,N-dimethylformamide (15 ml). The suspension was stirred under argon at 40° C. with successive additions of palladium (II) acetate (2×25 mg) occurring at 15 h and 23 h. The whole mixture was stirred at this temperature for a total time of 42 h. It was then cooled to ambient temperature and diluted with diethyl ether (40 ml) with stirring. The mixture was filtered and the filtrate evaporated to an oil, which was re-evaporated with toluene (50 ml). The residue from the evaporation was purified by column chromatography over silica gel, eluting with a solvent gradient of ethyl acetate/pentane to afford the title compound (D7) as a yellow solid (0.492 g, 1.5 mmol, 40%).

δH (CDCl₃, 250 MHz) 2.93 (2H, t, J=7.3 Hz), 3.55 (2H, t, J=7.3 Hz), 7.52-7.65 (4H, m), 7.76 (1H, dd, J=2, 8 Hz), 7.85 (1H, dd, J=2, 8 Hz), 8.0-8.08 (2H, m), 8.80 (1H, d, J=2 Hz), 9.25 (1H, d, J=2 Hz), 9.85 (1H, s).

Mass Spectrum: C₁₈H₁₅NO₃S requires 325; found 326 (MH⁺).

### Description 8

### 1-[3-(Phenylsulfonyl)quinolin-8-yl]ethanone

### D8

1-Ethoxyvinyl tri-butyl tin (0.97 ml, 3 mmol) was added to a stirred suspension of 8-iodo-3-phenylsulfonylquinoline (1.0 g, 2.5 mmol) in dry, degassed toluene (17 ml). To this suspension was added dichloro bis(triphenylphosphine) palladium (II) (88 mg, 125 umol) and the whole mixture was stirred at 105° C. for 66 h under argon. The mixture was then cooled to ambient temperature, concentrated in vacuo and the residue redissolved in tetrahydrofuran (25 ml). To this solution was added aqueous 2M hydrochloric acid (8 ml) and the solution was stirred for 5 h at ambient temperature. After dilution with dichloromethane (100 ml), the solution was washed with water (100 ml), dried (MgSO₄) and concentrated to an oil. The oil was purified by flash chromatography over silica gel eluting with dichloromethane. Fractions containing the product were pooled, concentrated and the resulting residue was stirred with diethyl ether (10 ml) to afford the title compound (D8) as a filterable yellow solid (0.532 g, 1.7 mmol, 68%).

δH (CDCl₃, 400 MHz) 2.90 (3H, s), 7.50-7.76 (4H, m), 8.04-8.12 (4H, m), 8.86 (1H, d, J=2.4 Hz), 9.32 (1H, d, J=2.4 Hz)

Mass Spectrum C₁₇H₁₃NO₃S requires 311; found 312 (MH⁺).

### Description 9

### 1-{1-[3-(Phenylsulfonyl)-8-quinolinyl]ethylidene}pyrrolidinium tetrafluoroborate

### D9

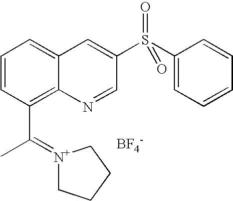

A suspension of 1-[3-(phenylsulfonyl)quinolin-8-yl]ethanone (D8) (220 mg, 0.71 mmol) pyrrolidinium tetrafluoroborate (102 mg, 0.71 mmol) in toluene (15 ml) was stirred at reflux for 18 h, whilst removing water by azeotropic distillation with the aid of a Dean-Stark water separator. After this time the mixture was cooled to ambient temperature to give an insoluble oil which was separated from the solvent by decantation. This oil was dried in vacuum at ambient temperature for 1 h and identified as the title compound (D9) (285 mg, 6.3 mmol, 89%).

δH (DMSO-d6, 250 MHz) 1.80-2.20 (4H, m), 3.33 (3H, s), 3.48-3.78 (2H, m), 4.12-4.45 (2H, m), 7.65-7.85 (3H, m), 7.96-8.20 (4H, m), 8.52-8.58 (1H, m), 9.40 (2H, s).

Mass Spectrum C₂₁H₂₁N₂O₂S. BF₄ cation requires 365; found 365 (M⁺).

### Description 10

### 8-Chloro-3-iodoquinoline

### D10

N-Iodosuccinimide (206.3 g, 0.92 mol) was added portionwise over 1 h to a stirred solution of 8-chloroquinoline (150 g, 0.92 mol) in acetic acid (750 ml) at 40° C. The reaction temperature was then increased to 65° C. and this was maintained for 18 h after which another portion of N-iodosuccinimide (61.9 g, 0.28 mmol) was added. After a further 4 h at this temperature, the mixture was cooled to ambient temperature and evaporated in vacuo to an oil. The oil was dissolved in dichloromethane (600 ml) and the solution was washed with saturated sodium thiosulfate solution (2×400 ml), dried (MgSO₄) and concentrated in vacuo to a solid (280 g). The solid was recrystallized from ethyl acetate (300 ml) to afford the title compound (D10) as a yellow solid (80 g). Concentration of the corresponding filtrate gave a second crop of title compound (30 g, total yield 45%). Mass Spectrum C₉H₅ CIIN requires 289; found 290 (MH⁺).

### Description 11

### 8-Chloro-3-[(4-fluorophenyl)thio]quinoline

### D11

Successive portionwise additions of potassium phosphate (102.7 g, 0.48 mol), copper (I) iodide (2.3 g, 12 mmol) and 8-chloro-3-iodoquinoline (D10) (70 g, 0.24 mol) were added with stirring to ethylene glycol (1 L) at ambient temperature. 4-Fluorobenzenethiol (38.6 ml, 0.363 mol) was added to the mixture in one portion and the whole was heated with stirring at 80° C. for 18 h. The mixture was then cooled to ambient temperature and water (800 ml) and dichloromethane (800 ml) were added. After vigorously stirring for 20 mins, the layers were separated and the stirred organic phase was treated with charcoal (20 g). After 0.5 h stirring, the mixture was filtered and the filtrate washed with water (500 ml), dried and concentrated in vacuo to afford the title compound (D11) as a crude yellow solid (78 g, 0.27 mol, 100%) which was used without purification in the next stage (see D12). Mass Spectrum C₁₅H₉CIFNS requires 289; found 290 (MH⁺).

### Description 12

### 8-Chloro-3-[(4-fluorophenyl)sulfonyl]quinoline

### D12

A solution of 8-chloro-3-[(4-fluorophenyl)thio]quinoline (D11) (70 g, nominal value 0.242 mol) in dichloromethane (200 ml) was added dropwise to a stirred mixture of monomagnesium peroxyphthalate hexahydrate (mmpp) (270 g, 0.545 mol) in dichloromethane (800 ml) and methanol (200 ml) at 0° C. After completed addition, the mixture was stirred for 48 h at ambient temperature. To this mixture was slowly added a 10% solution of sodium sulfite (500 ml) and the temperature kept below 30° C. whilst stirring for 0.5 h. The layers were separated and the organic phase was washed with saturated sodium hydrogen carbonate solution (2×300 ml) and concentrated in vacuo to a volume of approximately 300 ml. After cooling this solution in ice, the precipitated solid was filtered, washed with cold dichloromethane (200 ml), dried in vacuo at 35° C. for 12 h and identified as the title compound (D12) (30 g, 93.5 mmol, 39%). Mass Spectrum C₁₅H₉ClFNO₂S requires 321; found 322 (MH⁺).

### Description 13

### 8-Ethenyl-3-[(4-fluorophenyl)sulfonyl]quinoline

### D13

Successive additions of caesium fluoride (471 mg, 3.1 mmol), bis(tri-tert-butylphosphine)palladium (32 mg, 62 umol) and copper (I) iodide (12 mg, 62 umol) were made to a stirred solution of 8-chloro-3-[(4-fluorophenyl)sulfonyl]quinoline (D12) (0.5 g, 1.6 mmol) in N,N-dimethylformamide (4 ml) under argon and the mixture was heated at 80° C. for 24 h. After this time the mixture was cooled to ambient temperature, water (40 ml) and dichloromethane (100 ml) were added and the whole was shaken vigorously and then filtered through a pad of celite. The layers of the filtrate were separated and the organic phase dried (MgSO₄) and concentrated in vacuo to an oil. The oil was purified by chromatography over silica gel eluting with a gradient of ethyl acetate and hexane to afford the title compound (D13) (312 mg, 1 mmol, 64%). Mass Spectrum C₁₇H₁₂FNO₂S requires 313; found 314 (MH⁺).

### Description 14

### 8-[Bis(methyloxy)methyl]-3-[(4-fluorophenyl)sulfonyl]quinoline

### D14

The title compound (D14) was prepared from 8-ethenyl-3-[(4-fluorophenyl)sulfonyl]quinoline (D13) by ozonolysis as described in Description 2.

Mass Spectrum C₁₈H₁₆FNO₄S requires 361; found 362 (MH⁺).

### Description 15

### 3-[(4-Fluorophenyl)sulfonyl]-8-quinolinecarbaldehyde

### D15

The title compound (D15) was prepared from 8-[bis(methyloxy)methyl]-3-[(4-fluorophenyl)sulfonyl]quinoline (D14) by treatment with 1,2-dichlorethane and trifluoroacetic acid as described in Description 3. Mass Spectrum C₁₆H₁₀FNO₃S requires 315; found 316 (MH⁺).

### Description 16

### 8-(Bromomethyl)quinoline

### D16

8-Methylquinoline (Aldrich) (2.8 ml, 20.98 mmol) was dissolved in carbon tetrachloride (50 ml). N-Bromosuccimide (3.73 g, 20.98 mmol) and benzoyl peroxide 70%, remainder water (0.073 g, 0.2 mmol) were added and the reaction refluxed under argon overnight. After cooling the precipitate was filtered off. The filtrate was added to a stirred solution of aqueous sodium sulfite. The organics were separated and the aqueous was extracted with dichloromethane. The combined organics were washed with brine, dried over sodium sulfate, filtered and concentrated in vacuo to yield a brown solid (4.12 g). The residue was purified by column chromatography on a Biotage silica column (100 g) eluting with 5% ethyl acetate/n-hexane to yield the title compound as an off-white solid (1.76 g).

LC/MS [MH⁺] 222 consistent with molecular formula C₁₀H₈⁷⁹BrN.

### Description 17

### (3-Iodo-8-quinolinyl)methyl acetate

### D17

8-(Bromomethyl)quinoline (D16) (1.76 g, 7.93 mmol) was dissolved in acetic acid (20 ml) and N-iodosuccimide (2.68 g, 11.89 mmol) added and the reaction refluxed under argon for 2 hours. Further N-iodosuccimide (0.89 g, 3.96 mmol) was added and the reaction continued overnight. Further N-iodosuccimide (2.68 g, 11.89 mmol) was added and the reaction for an hour. Further N-iodosuccimide (1.79 g, 7.92 mmol) was added and the reaction continued for 2 hours. Aqueous sodium sulfite was added and the reaction stirred for 30 minutes. The reaction was further diluted with ethyl acetate and water and then carefully basified with saturated sodium bicarbonate solution to pH10. The organics were washed with saturated brine solution, dried over sodium sulfate, filtered and concentrated in vacuo to yield a dark oil (2.6 g). The residue was purified by column chromatography on a Biotage silica column (100 g) eluting with 80% dichloromethane/n-hexane to yield the title compound as an off-white solid (1.01 g).

LC/MS [MH⁺] 328 consistent with molecular formula C₁₂H₁₀INO₂.

### Description 18

### (3-Iodo-8-quinolinyl)methanol

### D18

(3-Iodo-8-quinolinyl)methyl acetate (D17) (1.01 g, 3.1 mmol) was dissolved in methanol (10 ml) and water (2 ml) and potassium hydroxide pellets (0.59 g, 10.5 mmol) added. The solution was refluxed under argon for 2 hours. The methanol was removed in vacuo and the residue diluted with ethyl acetate and water. The organics were separated and the aqueous extracted with 3 portions of ethyl acetate. The combined organics were washed with brine, dried over sodium sulfate, filtered and concentrated in vacuo to yield an off-white solid (0.86 g).

LC/MS [MH⁺] 286 consistent with molecular formula C₁₀H₈INO.

### Description 19

### 3-Iodo-8-quinolinecarbaldehyde

### D19

(3-Iodo-8-quinolinyl)methanol (D18) (0.45 g, 1.6 mmol), manganese (IV) oxide (1.72 g, 19.7 mmol) in dichloromethane (10 ml) was stirred at room temperature under argon for 4.5 hours. The reaction was filtered through a pad of celite and washed through with dichloromethane. The solvent was removed in vacuo to yield a white solid (420 mg). This was dissolved in hot methanol then allowed to cool to room temperature with scratching to yield white crystals (290 mg) which were filtered off.

¹H-NMR (400 MHz, CDCl₃) δ 7.73 (1H, t), 8.00 (1H, d), 8.35 (1H, d), 8.65 (1H, s), 9.18 (1H, s), 11.40 (1H, s).

### Description 20

### 8-[(3,3-Difluoro-1-piperidinyl)methyl]-3-iodoquinoline

### D20

3-Iodo-8-quinolinecarbaldehyde (D19) (2 g, 7.06 mmol) and 3,3-difluoropiperidine hydrochloride (0.94 g, 7.77 mmol) were dissolved in dichloromethane (20 ml). Sodium triacetoxyborohydride (2.25 g, 10.6 mmol) and acetic acid (0.44 ml, 7.77 mmol) were added and the reaction stirred at room temperature under argon overnight. The reaction was diluted with dichloromethane, cooled and basified with saturated sodium bicarbonate solution. The organics were dried over sodium sulfate, filtered and concentrated in vacuo to yield a brown oil (2.89 g). The residue was purified on the Jones Flashmaster II with an IST SPE 70 g silica column eluting a gradient of 5-12% ethyl acetate/n-hexane over 40 minutes. This yielded the title compound as a pale yellow oil (2.27 g).

LC/MS [MH⁺] 389 consistent with molecular formula C₁₅H₁₅F₂IN₂.

### EXAMPLE 1

### 3-(Phenylsulfonyl)-8-(1-pyrrolidinylmethyl)quinoline hydrochloride

### E1

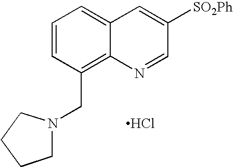

A suspension of 3-(phenylsulfonyl)-8-quinolinecarbaldehyde (D3) (95 mg, 0.32 mmol) in 1,2-dichloroethane (2 ml) was added over 2 mins to a stirred mixture of pyrrolidine (0.029 ml, 0.35 mmol), acetic acid (0.009 ml, 0.16 mmol) and sodium triacetoxyborohydride (74 mg, 0.35 mmol) in 1,2-dichloroethane (1 ml) under argon at ambient temperature. After stirring for 18 h, the mixture was diluted with 1,2-dichloroethane (20 ml) and washed with 0.5M sodium hydroxide solution (10 ml) then water (10 ml), dried (MgSO₄) and evaporated in vacuo to an oil. This material was passed through a solid phase cartridge (SCX) eluting sequentially with dichloromethane, methanol and a mixture of methanol/ammonium hydroxide solution, d=0.88 (10:1). Fractions collected from the latter eluant system were combined and evaporated in vacuo to an oil. The oil was dissolved in dichloromethane (1 ml) and a 1M solution of hydrogen chloride in diethyl ether (0.5 ml) was added to afford the title compound (E1) as a solid (48 mg, 0.12 mmol, 39%).

δH (DMSO-d₆, 250 MHz) 1.73-2.09 (4H, m), 3.13-3.38 (4H, m), 4.93 (2H, d, J=5.7 Hz), 7.68-7.75 (3H, m), 7.88 (1H, t, J=8.0 Hz), 8.14-8.18 (2H, m), 8.30 (1H, dd, J=2.5, 8 Hz), 8.42 (1H, dd, J=2.5, 8 Hz), 9.31 (1H, d, J=2.5 Hz), 9.40 (1H, J=2.5 Hz), 10.2 (1H, br, s).

Mass Spectrum: C₂₀H₂₀N₂O₂S requires 352; found 353 (MH⁺)

### EXAMPLES 2-9

### E2-9

Examples 2-9 were prepared as hydrochloride salts by treatment of 3-(phenylsulfonyl)-8-quinolinecarbaldehyde (D3) with the appropriate amine, as described in Example 1.

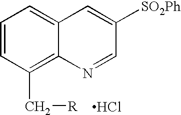

| | | | | [MH]⁺ |
|---|---|---|---|---|
| Example | | | δH (DMSO-d₆, | (mol. |
| No. | Chemical Name | R | 250 MHz) | Formula) |
| 2 | 3-(Phenylsulfonyl)-8-(1-piperidinylmethyl)quinoline | 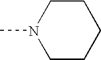 | 1.30-1.85 (6H, m),2.90-3.10 (4H, m),4.86 (2H, d, J =5.3 Hz), 7.64-7.75 (3H,m), 7.89 (1H, t, J =7.3 Hz), 8.14-8.18 (2H,m), 8.35 (1H, dd, J =2.5, 8 Hz), 8.43 (1H,dd, J = 2.5, 8 Hz), 9.30(1H, d, J = 2.5 Hz),9.41 (1H, d, J =2.5 Hz), 9.7 (1H, br, s). | 367(C₂₁H₂₂N₂O₂S) |
| 3 | 8-[(4-Methyl-1-piperazinyl)methyl]-3-(phenylsulfonyl)quinoline | 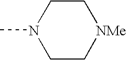 | 2.78 (3H, s), 3.2-4.3(8H, m, partiallymasked by HOD), 4.7-4.9 (2H, br, s), 7.64-7.74 (3H, m), 7.88 (1H,t, J = 7.5 Hz), 8.12-8.18(2H, m), 8.26-8.40 (2H,m), 9.28 (1H, d, J =2.5 Hz), 9.38 (1H, d, J =2.5 Hz), 11.1 (1H, br, s). | 382(C₂₁H₂₃N₃O₂S) |
| 4 | 8-(4-(Morpholinylmethyl)-3-(phenylsulfonyl)quinoline | 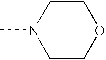 | 3.20-3.36 (4H, m),3.66-3.96 (4H, m,partially masked byHOD), 4.94 (2H, d, J =3.9 Hz), 7.66-7.80 (3H,m), 7.90 (1H, t, J =8.0 Hz), 8.12-8.18 (2H,m), 8.30 (1H, dd, J = 2,8 Hz), 8.42 (1H, dd, J =2, 8 Hz), 9.32 (1H, d, J =2 Hz), 9.40 (1H, d, J =2 Hz), 10.4 (1H, br, s). | 369(C₂₀H₂₀N₂O₃S) |
| 5 | 8-[(4,4-Difluoro-1-piperidinyl)methyl]-3-(phenylsulfonyl)quinoline | 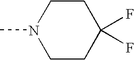 | 2.25-2.40 (4H, m),3.20-3.58 (4H, m),4.99 (2H, s), 7.76-7.80(3H, m), 7.90 (1H, t, J =8.0 Hz), 8.10-8.18(2H, m), 8.38 (1H, dd,J = 2, 8 Hz), 8.46 (1H,dd, J = 2, 8 Hz), 9.32(1H, d, J = 2 Hz), 9.40(1H, d, J = 2 Hz), 10.8(1H, br, s). | 403(C₂₁H₂₀F₂N₂O₂S) |
| 6 | 8-[(2,5-Dimethyl-1-pyrrolidinyl)methyl)-3-(phenylsulfonyl)quinoline | 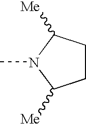 | — | 381(C₂₂H₂₄N₂O₂S) |
| 7 | N,N-Dimethyl-1-[3-(phenylsulfonyl)quinolin-8-yl]methanamine | —NMe₂ | — | 327(C₁₈H₁₈N₂O₂S) |
| 8 | 8-[(2-Methylpyrrolidin-1-yl)methyl]-3-(phenylsulfonyl)quinoline | 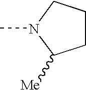 | — | 367(C₂₁H₂₂N₂O₂S) |
| 9 | N-Isopropyl-N-{[3-(phenylsulfonyl)quinoiin-8-yl]methyl}propan-2-amine | —N(i-Pr)₂ | — | 383(C₂₂H₂₆N₂O₂S) |

### EXAMPLE 10

### 3-(Phenylsulfonyl)-8-[2-(1-pyrrolidinyl)ethylquinoline hydrochloride

### E10

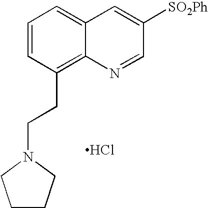

To a solution of crude [3-(phenylsulfonyl)-8-quinolinyl]acetaldehyde (D6) (0.118 g, 0.38 mmol) in 1,2-dichloroethane (5 ml) was added pyrrolidine (0.07 ml, 0.84 mmol) and acetic acid (0.011 ml, 0.19 mmol). After stirring the solution for 1 h at ambient temperature, sodium triacetoxyborohydride (89 mg, 0.42 mmol) was added and the solution was stirred for a further 18 h. The solution was then diluted with 1,2-dichloroethane (10 ml) and washed with 0.5M sodium hydroxide solution (5 ml) then water (5 ml), dried (MgSO₄) and evaporated in vacuo to an oil. The material was purified by column chromatography over silica gel eluting with a solvent gradient system of dichloromethane/methanol/methanol-ammonium hydroxide solution, d=0.88 (10:1) to give an oil. This oil was dissolved in dichloromethane (1 ml) and diluted with a 1M solution of hydrogen chloride in diethyl ether (0.5 ml) to afford the title compound (E10) as a solid (12 mg, 0.03 mmol, 8%).

δH (DMSO-d₆, 250 MHz) 1.82-2.08 (4H, m), 3.02-3.14 (2H, m), 3.45-3.64 (6H, m), 7.66-7.80 (3H, m), 7.95 (1H, dd, J=2, 8 Hz), 8.10-8.13 (2H, m), 8.24 (1H, dd, J=2, 8 Hz), 8.42 (1H, dd, J=2.5, 8 Hz), 9.23 (1H, d, J=2 Hz), 9.35 (1H, d, J=2 Hz), 10.2 (1H, br, s).

Mass Spectrum: C₂₁H₂₂N₂O₂S requires 366; found 367 (MH⁺)

### EXAMPLE 11

### 3-(Phenylsulfonyl)-8-[2-(1-piperidiny)ethyl]quinoline hydrochloride

### E11

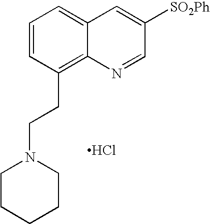

The title compound (E11) was prepared in 13% yield as described in Example 10, using [3-(phenylsulfonyl)-8-quinolinyl]acetaldehyde (D6) and piperidine.

δH (DMSO-d₆, 250 MHz) 1.35-1.90 (6H, m), 2.89-3.04 (2H, m), 3.30-3.40 (2H, m), 3.50-3.70 (4H, m, partially masked by HOD), 7.65-7.80 (4H, m), 7.95 (1H, dd, J=2, 8 Hz), 8.06-8.14 (2H, m), 8.23 (1H, dd, J=2, 8 Hz), 9.21 (1H, d, J=2 Hz), 9.34 (1H, d, J=2 Hz), 9.95 (1H, br, s).

Mass Spectrum: C₂₂H₂₄N₂O₂S requires 380; found 381 (MH⁺)

### EXAMPLE 12

### 3-(Phenylsulfonyl)-8-[3-(1-pyrrolidinyl)propyl]quinoline hydrochloride

### E12

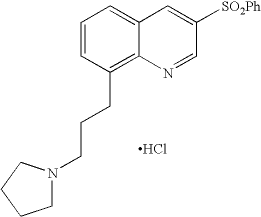

The title compound (E12) was prepared in 22% yield as described in Example 1, using 3-[3-(phenylsulfonyl)-8-quinolinyl]propanal (D7) and pyrrolidine.

δH (DMSO-d₆, 250 MHz) 1.80-2.15 (6H, m), 2.88-3.00 (2H, m), 3.10-3.28 (4H, m), 3.40-3.58 (2H, m, partially masked by HOD), 7.62-7.79 (4H, m), 7.90 (1H, d, J=5.9 Hz), 8.10-8.18 (3H, m), 9.20 (1H, d, J=2 Hz), 9.35 (1H, d, J=2 Hz), 10.1 (1H, br, s).

Mass Spectrum: C₂₂H₂₄N₂O₂S requires 380; found 381 (MH⁺)

### EXAMPLES 13-15

### E13-E15

Examples 13-15 were prepared as hydrochloride salts by treatment of 3-[3-(phenylsulfonyl)-8-quinolinyl]propanal (D7) with the appropriate amine, as described in Example 1.

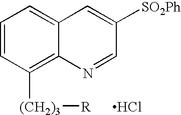

| | | | | [MH]⁺ |
|---|---|---|---|---|
| Example | | | δH (DMSO-d₆, | (mol. |
| No. | Chemical Name | R | 250 MHz) | Formula) |
| 13 | 3-(Phenylsulfonyl)-8-[3-(1-piperidinyl)propyl]quinoline | 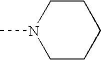 | 1.30-2.15 (8H, m),2.75-3.45 (8H, m),7.65-7.80 (4H, m),7.90-8.20 (4H, m),9.18 (1H, d, J = 2 Hz),9.33 (1H, d, J = 2 Hz),9.35 (1H, br, s). | 395(C₂₃H₂₆N₂O₂S) |
| 14 | 8-[3-(4-(Morpholinyl)propyl]-3-(phenylsulfonyl)quinoline | 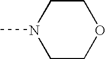 | 2.08-2.18 (2H, m),2.95-3.45 (8H, m),3.70-3.97 (4H, m),7.65-7.80 (4H, m),7.90 (1H, d, J =6.0 Hz), 8.15-8.20 (3H,m), 9.20 (1H, d, J =2 Hz), 9.35 (1H, d, J =2 Hz), 10.7 (1H, br, s). | 397(C₂₂H₂₄N₂O₃S) |
| 15 | N,N-Dimethyl-3-[3-(phenylsulfonyl)-8-quinolinyl]-1-propanamine | —NMe₂ | 2.00-2.12 (2H, m),2.73 (6H, d, J =4.7 Hz), 3.04-3.15 (2H,m), 3.23 (2H, t, J =7.5 Hz), 7.65-7.79 (4H,m), 7.90 (1H, d, J =8 Hz), 8.11-8.20 (3H,m), 9.20 (1H, d, J =2 Hz), 9.35 (1H, d, J =2 Hz), 9.75 (1H, br, s). | 355(C₂₀H₂₂N₂O₂S) |

### EXAMPLE 16

### 3-(Phenylsulfonyl)-8-(1-pyrrolidin-1-ylethyl)quinoline hydrochloride

### E16

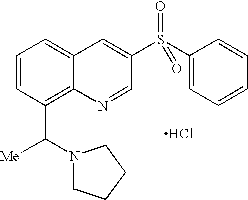

A solution of 1-[3-(phenylsulfonyl)quinolin-8-yl]ethanone (D8) (25 mg, 88 umol) in 1,2-dichloroethane (0.5 ml) was added dropwise to a stirred mixture of pyrrolidine (7.3 uL, 88 umol), sodium triacetoxyborohydride (19 mg, 88 umol) and acetic acid (2.3 uL, 40 umol) in 1,2-dichloroethane (0.5 ml) and left to stir under argon overnight. After this time, more pyrrolidine (7.3 uL, 88 umol) and sodium triacetoxyborohydride (19 mg, 88 umol) were added to the mixture which was stirred overnight. Another addition of pyrrolidine (7.3 uL, 88 umol) and sodium triacetoxyborohydride (19 mg, 88 umol) were made and the mixture left to stir for another 24 h. To the mixture was then added 1,2-dichloroethane (10 ml) and the solution was washed successively with 0.5M aqueous sodium hydroxide (10 ml) and water (10 ml), then dried (MgSO₄) and concentrated to an oil. The oil was dissolved in dichloromethane (2 ml) and diluted successively with 1M hydrogen chloride in diethyl ether (0.1 ml) and diethyl ether to afford the title compound (E16) as a solid (16 mg, 40 umol, 50%).

δH (DMSO-d6, 250 MHz) 1.72 (3H, d, J=6.8 Hz), 1.75-2.10 (4H, m), 2.65-2.80 (1H, m), 2.92-3.04 (1H, m), 3.29-3.40 (1H, m), 3.72-3.84 (1H, m), 5.60-5.70 (1H, m), 7.65-7.80 (3H, m), 7.90-7.95 (1H, m), 8.11-8.18 (2H, m), 8.32-8.40 (1H, m), 8.50-8.55 (1H, m), 9.30-9.38 (2H, m), 10.0 (1H, br, s).

Mass Spectrum C₂₁H₂₂N₂O₂S requires 366; found 367 (MH⁺).

### EXAMPLES 17-19

### E17-19

Examples 17-19 were prepared as hydrochloride salts by treatment of 1-[3-(phenylsulfonyl)quinolin-8-yl]ethanone (D8) with the appropriate amine, as described in Example 16.

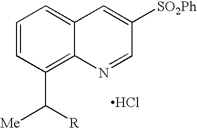

| | | | [MH]⁺ |
|---|---|---|---|
| Example | | | (mol. |
| No. | Chemical Name | R | Formula) |
| 17 | 3-(phenylsulfonyl)-8-(1-piperidin-1-ylethyl)quinoline | 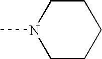 | 381(C₂₂H₂₄N₂O₂S) |
| 18 | 8-(1-morpholin-4-ylethyl)-3-(phenylsulfonyl)-quinoline | 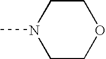 | 383(C₂₁H₂₂N₂O₃S) |
| 19 | N,N-dimethyl-1-[3-(phenylsulfonyl)quinolin-8-yl]ethanamine | —NMe₂ | 341(C₁₉H₂₀N₂O₂S) |

### EXAMPLE 20

### N-Methyl-N-{1-[3-(phenylsulfonyl)quinolin-8-yl]ethyl}propan-2-amine hydrochloride

### E20

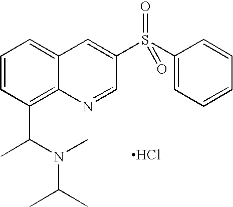

Titanium (IV) isopropoxide (176 uL, 0.6 mmol) was added to a stirred solution of 1-[3-(phenylsulfonyl)quinolin-8-yl]ethanone (D8) (150 mg, 0.48 mmol) and isopropyl(methyl)amine (75 uL, 0.72 mmol) in dry tetrahydrofuran (2 ml) at ambient temperature. After 1 h, sodium triacetoxyborohydride (153 mg, 0.72 mmol) was added and the mixture was continued to stir for 4 h. The mixture was then diluted with dichloromethane (10 ml), washed with 0.2M aqueous sodium hydroxide (10 ml) and the organic phase dried (MgSO₄) and concentrate in vacuo to an oil. The crude oil was purified by mass directed auto-preparative chromatography to give material which was treated with 1M hydrogen chloride in diethyl ether to afford the title compound (E20) (45 mg, 0.11 mmol, 23%). Mass Spectrum C₂₁H₂₄N₂O₂S requires 368; found 369 (MH⁺).

### EXAMPLE 21

### N-Ethyl-N-methyl-1-[3-(phenylsulfonyl)quinolin-8-yl]ethanamine hydrochloride

### E21

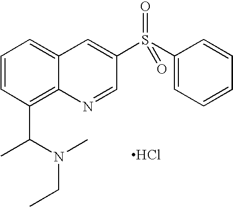

The title compound (E21) (55% yield) was prepared from 1-[3-(phenylsulfonyl)quinolin-8-yl]ethanone (D8) and ethyl(methyl)amine as described in Example 20. Mass Spectrum C₂₀H₂₂N₂O₂S requires 354; found 355 (MH⁺).

### EXAMPLE 22

### 8-(1-Methyl-1-pyrrolidin-1-ylethyl)-3-(phenylsulfonyl)quinoline hydrochloride

### E22

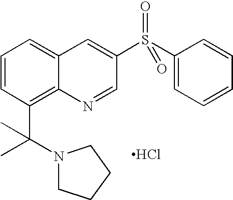

Cerium chloride heptahydrate (510 mg, 1.35 mmol) was heated at 140° C. under vacuum (1 mm Hg pressure) for 3 h in a Schlenk tube. After cooling to ambient temperature, dry tetrahydrofuran (2 ml) was added under argon and the suspension was sonicated for 1 h and subsequently stirred for 2 h. This suspension was added under argon to a stirred suspension of 1-{1-[3-(phenylsulfonyl)-8-quinolinyl]ethylidene}pyrrolidinium tetrafluoroborate (D9) (124 mg, 0.27 mmol) in dry tetrahydrofuran (1 ml) and cooled to −30° C. To this suspension was added dropwise methyl magnesium bromide solution (1.4M in tetrahydrofuran) (0.96 ml, 1.35 mmol) and stirred at this temperature for 2 h and subsequently slowly warmed to ambient temperature overnight. The mixture was poured into saturated sodium hydrogen carbonate solution (25 ml) and then extracted with ethyl acetate (2×25 ml). The combined organic extracts were dried (MgSO4) and concentrated in vacuo to an oil. The oil was purified by mass directed auto-preparative chromatography eluting with a solvent gradient containing formic acid to give the title compound as the formate salt (6.3 mg, 16 umol, 6%).

δH (CDCl₃, 250 MHz) 1.94 (6H, s), 2.05-2.12 (4H, m), 3.35-3.48 (4H, m), 7.60-7.80 (4H, m), 7.94-8.0 (1H, m), 8.05-8.12 (3H, m), 8.38 (1H, br, s), 8.95 (1H, d, J=2.3 Hz), 9.39 (1H, d, J=2.3 Hz).

Mass Spectrum C₂₂H₂₄N₂O₂S requires 380; found 381 (MH⁺).

This material was converted to the hydrochloride salt (6.6 mg) by treatment with hydrogen chloride in diethyl ether.

Mass Spectrum C₂₂H₂₄N₂O₂S requires 380; found 381 (MH⁺).

### EXAMPLE 23

### 3-[(4-Fluorophenyl)sulfonyl]-8-(1-pyrrolidinylmethyl)quinoline hydrochloride

### E23

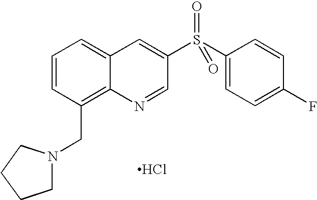

The title compound (E23) was prepared from 3-[(4-fluorophenyl)sulfonyl]-8-quinolinecarbaldehyde (D15) as described in Example 1.

Mass Spectrum C₂₀H₁₉FN₂O₂S requires 370; found 371 (MH⁺).

### EXAMPLE 24

### 8-[(4-acetyl-1-piperazinyl)methyl]-3-(phenylsulfonyl)quinoline hydrochloride

### E24

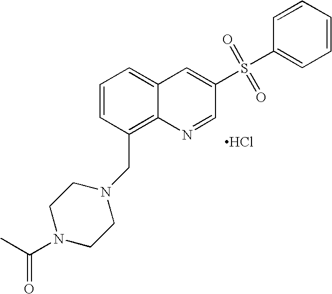

The title compound (E24) was prepared from 3-(phenylsulfonyl)-8-quinolinecarboxaldhyde (D3) and N-acetylpiperazine, in a similar manner to that described in Example 1, omitting the SCX purification step.

Mass spectrum (ES) (C22H23N3O3S, MH+ 410)

### EXAMPLE 25

N-{[3-(phenylsulfonyl)-8-quinolinyl]methyl}-2-propanamine hydrochloride

### E25

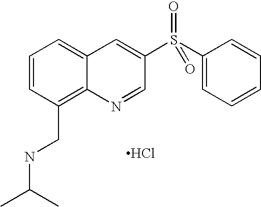

The title compound (E25) was prepared from 3-(phenylsulfonyl)-8-quinolinecarboxaldhyde (D3) and 2-propanamine, in a similar manner to that described in Example 1, omitting the SCX purification step.

Mass Spectrum (ES) (C19H20N2O2S, MH+ 341)

### EXAMPLE 26

### N-methyl-1-[3-(phenylsulfonyl)-8-quinolinyl]methanamine hydrochloride

### E26

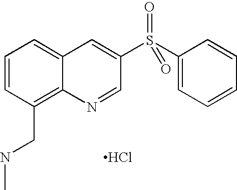

The title compound (E26) was prepared from 3-(phenylsulfonyl)-8-quinolinecarboxaldhyde (D3) and a solution of methylamine in ethanol, in a similar manner to that described in Example 1, omitting the SCX purification step. Purification of crude free base material was performed by mass-directed auto-preparative chromatography using a 10 minute gradient containing water and between 15% and 55% acetonitrile with 0.1% formic acid. Product fractions were collected, evaporated to a gum and converted to the hydrochloride salt as described in Example 1.

Mass spectrum (C17H16N2O2S, MH+ 313)

### EXAMPLE 27

### 8-[(3,3-Difluoropiperidin-1-yl)methyl]-3-(phenylsulfonyl)quinoline hydrochloride

### E27

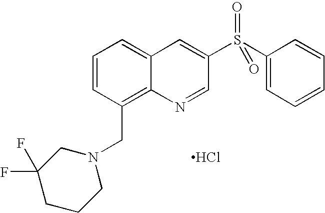

A suspension of 3-(phenylsulfonyl)quinoline-8-carbaldehyde (D3) (45 mg, 0.15 mmol) in anhydrous dichloromethane (1 ml) was treated with 3,3-difluoropiperidine hydrochloride (26 mg, 0.165 mmol) and sodium triacetoxyborohydride (37 mg, 0.175 mmol) and the mixture was stirred under argon at room temperature for 18 h. The reaction mixture was then diluted with dichloromethane (30 ml) and washed with aqueous sodium bicarbonate solution (2×20 ml). The dichloromethane solution was dried by filtration through a hydrophobic cartridge and evaporated to a gum. This material was dissolved in a mixture of dimethylsulphoxide (0.45 ml) and acetonitrile (0.45 ml) and purified by mass-directed auto-preparative chromatography using a 10 minute gradient containing water and between 15% and 55% acetonitrile with 0.1% formic acid. Product fractions were collected and evaporated to a gum. This material was dissolved in ether (2 ml) and treated with 1M hydrogen chloride in ether (1 ml). The mixture was evaporated, dissolved in ether and re-evaporated to yield the title compound as a white solid (35 mg, 0.08 mmol, 53%).

δH (CD₃OD, 400 MHz) 1.90-2.40 (4H, m), 3.20-3.90 (4H, m), 5.06 (2H, s), 7.61-7.72 (3H, m), 7.86 (1H, t, J=7 Hz), 8.06-8.16 (3H, m), 8.34 (1H, dd, J=1.2, 8.4 Hz), 9.17 (1H, d, J=2 Hz), 9.40 (1H, d, J=2 Hz).

Mass spectrum: C₂₁H₂₀F₂N₂O₂S requires 402; found 403 (MH⁺)

### EXAMPLE 28

### 8-[(4-Methoxypiperidin-1-yl)methyl]-3-(phenylsulfonyl)quinoline hydrochloride

### E28

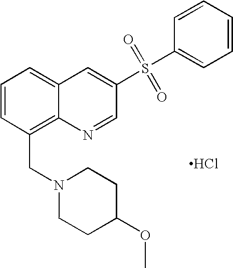

Using the procedure described in Example 27 with 3-(phenylsulfonyl)quinoline-8-carbaldehyde (D3) (45 mg, 0.15 mmol) and 4-methoxypiperidine hydrochloride salt (25 mg, 0.165 mmol), the title compound was obtained as a white solid (25 mg, 0.06 mmol, 42%).

δH (CD₃OD, 400 MHz) 1.50-2.30 (4H, m), 3.20-3.60 (4H, m), 3.35 (3H, s), 4.97 (2H, s), 7.62-7.72 (3H, m), 7.84 (1H, t, J=8 Hz), 8.11-8.20 (3H, m), 8.32 (1H, d, J=8.4 Hz), 9.16 (1H, d, J=2 Hz), 9.40 (1H, d, J=2 Hz).

Mass spectrum: C₂₂H₂₄N₂O₃S requires 396; found 397 (MH⁺)

### EXAMPLE 29

### 8-[(2-Methyl-1-piperidinyl)methyl]-3-(phenylsulfonyl)quinoline hydrochloride

### E29

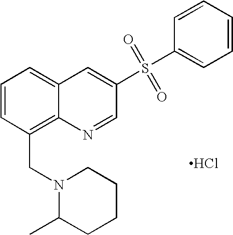

Using the procedure described in Example 27 with 3-(phenylsulfonyl)quinoline-8-carbaldehyde (D3) (45 mg, 0.15 mmol) and 2-methylpiperidine (17 mg, 0.17 mmol) and acetic acid (0.005 ml), the title compound was obtained as a white solid (20 mg, 32%).

δH (DMSO-d6, 400 MHz) includes 7.64-7.81 (3H, m), 7.87 (1H, t, J=8 Hz), 8.09-8.13 (2H, m), 8.22-8.27 (1H, m), 8.41-8.43 (1H, m), 9.31 (1H, m), 9.36 (1H, m)

Mass spectrum: C₂₀H₂₄N₂O₂S requires 380; found 381 (MH⁺).

### EXAMPLE 30

### 8-[(3-Methyl-1-piperidinyl)methyl]-3-(phenylsulfonyl)quinoline hydrochloride

### E30

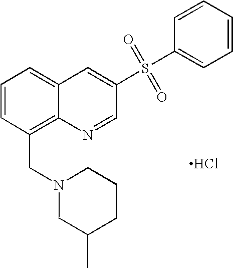

Using the procedure described in Example 27 with 3-(phenylsulfonyl)quinoline-8-carbaldehyde (D3) (45 mg, 0.15 mmol) and 3-methylpiperidine (17 mg, 0.17 mmol) and acetic acid (0.005 ml), the title compound was obtained as a white solid (20 mg, 32%).

δH (CD₃OD, 400 MHz) 0.95 (3H, d, J=6 Hz), 1.13 (1H, m), 1.57-2.03 (4H, m), 2.76 (1H, m), 3.01 (1H, m), 3.40-3.59 (2H, m), 7.61-7.72 (3H, m), 7.84 (1H, t, J=8 Hz), 8.08-8.13 (3H, m), 8.32 (1H, d, J=8 Hz), 9.16 (1H, d, J=2 Hz), 9.40 (1H, d, J=2 Hz)

Mass spectrum: C₂₀H₂₄N₂O₂S requires 380; found 381 (MH⁺)

### EXAMPLE 31

### 8-(1,4-Dioxa-8-azaspiro[4.5]dec-8-ylmethyl)-3-(phenylsulfonyl)quinoline hydrochloride

### E31

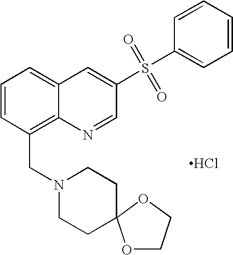

Using the procedure described in Example 27 with 3-(phenylsulfonyl)quinoline-8-carbaldehyde (D3) (45 mg, 0.15 mmol) and 1,4-dioxa-8-azaspiro[4.5]decane (24 mg, 0.165 mmol) and acetic acid (0.005 ml), the title compound was obtained as a cream solid (28 mg, 40%).

δH (CD₃OD, 400 MHz) 1.96-1.98 (4H, m), 3.30-3.58 (4H, m), 3.99 (4H, s), 4.95 (2H, s), 7.63-7.65 (3H, m), 7.84 (1H, t, J=8 Hz), 8.10-8.13 (3H, m), 8.30 (1H, d, J=8 Hz), 9.16 (1H, d, J=2 Hz), 9.38 (1H, d, J=2 Hz).

Mass spectrum: C₂₃H₂₄N₂O₄S requires 424; found 425 (MH⁺)

### EXAMPLE 32

### 8-[(1,1-Dioxido-4-thiomorpholinyl)methyl]-3-(phenylsulfonyl)quinoline hydrochloride

### E32

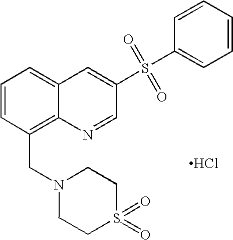

Using the procedure described in Example 27 with 3-(phenylsulfonyl)quinoline-8-carbaldehyde (D3) (45 mg, 0.15 mmol) and thiomorpholine 1,1-dioxide hydrochloride salt (28 mg, 0.165 mmol), the title compound was obtained as a white solid (30 mg, 48%) prior to salt formation with hydrogen chloride in ether.

δH (CDCl₃, 400 MHz) 3.08-3.13 (8H, m), 4.39 (2H, s), 7.52-7.64 (3H, m), 7.69 (1H, t, J=8 Hz), 7.91-8.06 (4H, m), 8.82 (1H, d, J=2.4 Hz), 9.25 (1H, d, J=2.4 Hz).

Mass spectrum: C₂₀H₂₀N₂O₄S₂ requires 416; found 417 (MH⁺)

### EXAMPLE 33

### 8-[(3,3-Difluoro-1-pyrrolidinyl)methyl]-3-(phenylsulfonyl)quinoline hydrochloride

### E33

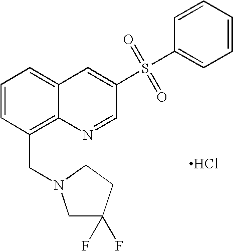

Using the procedure described in Example 27 with 3-(phenylsulfonyl)quinoline-8-carbaldehyde (D3) (45 mg, 0.15 mmol) and 3,3-difluoropyrrolidine hydrochloride (24 mg, 0.165 mmol), the title compound was obtained as a white solid (42 mg, 66%).

δH (CD₃OD, 400 MHz) 2.60-2.80 (2H, m), 3.76-3.78 (2H, m), 3.97 (2H, t, J=12 Hz), 5.10 (2H, s), 7.61-7.72 (3H, m), 7.85 (1H, t, J=7 Hz), 8.06-8.16 (3H, m), 8.34 (1H, dd, J=1.2, 8.4 Hz), 9.18 (1H, d, J=2 Hz), 9.41 (1H, d, J=2 Hz)

Mass spectrum: C₂₀H₁₈F₂N₂O₂S requires 388; found 389 (MH⁺)

### EXAMPLE 34

### 8-[(3,3-Difluoro-1-azetidinyl)methyl]-3-(phenylsulfonyl)quinoline hydrochloride

### E34

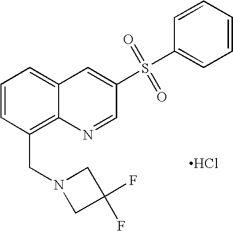

Using the procedure described in Example 27 with 3-(phenylsulfonyl)quinoline-8-carbaldehyde (D3) (45 mg, 0.15 mmol) and 3,3-difluoroazetidine hydrochloride (22 mg, 0.165 mmol), the title compound was obtained as a white solid (31 mg, 50%).

δH (CD₃OD, 400 MHz) 4.77-4.82 (4H, m), 5.10 (2H, s), 7.61-7.72 (3H, m), 7.83 (1H, t, J=8 Hz), 8.10-8.13 (3H, m), 8.31 (1H, dd, J=1.2, 8.4 Hz), 9.16 (1H, d, J=2 Hz), 9.40 (1H, d, J=2 Hz)

Mass spectrum: C₁₉H₁₆F₂N₂O₂S requires 374; found 375 (MH⁺)

### EXAMPLE 35

### 8-[1-(3,3-Difluoro-1-azetidinyl)ethyl]-3-(phenylsulfonyl)quinoline

### E35

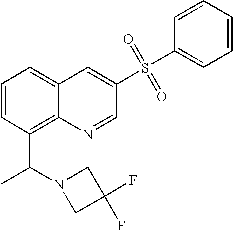

Using the procedure described in Example 27 with 1-[3-(phenylsulfonyl)quinolin-8-yl]ethanone (D8) (48 mg, 0.15 mmol) and 3,3-difluoroazetidine hydrochloride (22 mg, 0.165 mmol), the title compound was obtained as a white solid (30 mg, 52%) prior to salt formation with hydrogen chloride in ether.

δH (CDCl₃, 400 MHz) 1.35 (3H, t, J=7 Hz), 3.53-3.66 (4H, m), 4.89 (1H, q, J=7 Hz), 7.53-7.63 (3H, m), 7.69 (1H, t, J=8 Hz), 7.87 (1H, dd, J=1.6, 8 Hz), 8.04-8.07 (3H, m), 8.81 (1H, d, J=2 Hz), 9.26 (1H, d, J=2 Hz)

Mass spectrum: C₂₀H₁₈F₂N₂O₂S requires 388; found 389 (MH⁺)

### EXAMPLE 36

### 8-[1-(3,3-Difluoro-1-pyrrolidinyl)ethyl]-3-(phenylsulfonyl)quinoline

### E36

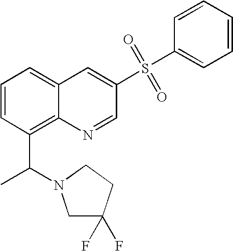

Using the procedure described in Example 27 with 1-[3-(phenylsulfonyl)quinolin-8-yl]ethanone (D8) (48 mg, 0.15 mmol) and 3,3-difluoropyrrolidine hydrochloride (24 mg, 0.165 mmol), the title compound was obtained as a white solid (35 mg, 58%) prior to salt formation with hydrogen chloride in ether.

δH (CDCl₃, 400 MHz) 1.41 (3H, t, J=7 Hz), 2.22-2.33 (2H, m), 2.68 (1H, q, J=8 Hz), 2.85-2.95 (3H, m), 4.86 (1H, q, J=7 Hz), 7.54-7.63 (3H, m), 7.71 (1H, t, J=8 Hz), 7.87 (1H, d, J=8 Hz), 8.05 (2H, d, J=8 Hz), 8.13 (1H, d, J=7 Hz), 8.81 (1H, d, J=2 Hz), 9.25 (1H, d, J=2 Hz)

Mass spectrum: C₂₁H₂₀F₂N₂O₂S requires 402; found 403 (MH⁺)

### EXAMPLE 37

### 8-[1-(3,3-Difluoro-1-piperidinyl)ethyl]-3-(phenylsulfonyl)quinoline

### E37

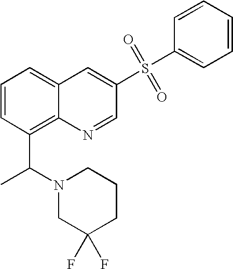

A suspension of 1-[3-(phenylsulfonyl)quinolin-8-yl]ethanone (D8) (96 mg, 0.3 mmol) in anhydrous dichloromethane (2 ml) was treated with 3,3-difluoropiperidine hydrochloride (52 mg, 0.33 mmol) and sodium triacetoxyborohydride (95 mg, 0.44 mmol) and the mixture was stirred under argon at room temperature for 3 days. The reaction mixture was then diluted with dichloromethane (75 ml) and washed with aqueous sodium bicarbonate solution (2×50 ml). The dichloromethane solution was dried by filtration through a hydrophobic cartridge and evaporated to a gum. This material was dissolved in a mixture of dimethylsulphoxide (0.90 ml) and acetonitrile (0.90 ml) and purified by mass-directed auto-preparative chromatography using a 10 minute gradient containing water and between 15% and 55% acetonitrile with 0.1% formic acid. Product fractions were collected and evaporated to give the title compound as a colourless foam (14 mg, 11%).

δH (CDCl₃, 400 MHz) 1.41 (3H, t, J=7 Hz), 1.70-1.89 (4H, m), 2.33-2.36 (1H, m), 2.61-2.78 (3H, m), 5.00 (1H, q, J=7 Hz), 7.52-7.63 (3H, m), 7.70 (1H, t, J=8 Hz), 7.86 (1H, dd, J=1.2 Hz, 7 Hz), 7.99-8.10 (3H, m), 8.81 (1H, d, J=2 Hz), 9.24 (1H, d, J=2 Hz)

Mass spectrum: C₂₂H₂₂F₂N₂O₂S requires 416; found 417 (MH⁺)

### EXAMPLE 38

### 8-[(3,3-Difluoro-1-piperidinyl)methyl]-3-[(4-fluorophenyl)sulfonyl]quinoline hydrochloride

### E38

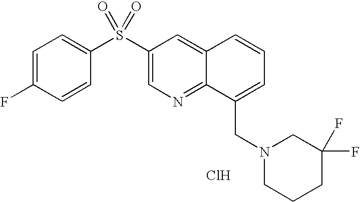

8-[(3,3-Difluoro-1-piperidinyl)methyl]-3-iodoquinoline (0.1 g, 0.26 mmol), sodium 4-fluorophenylsulfinate (0.095 g, 0.52 mmol), potassium carbonate (36 mg, 0.26 mmol), copper triflate (0.009 g, 0.026 mmol) were dissolved in dimethylsulfoxide (2 ml) and N,N-dimethylethylenediamine (5.59 μl, 0.052 mmol) was added. The reaction mixture was heated to 100° C. and stirred under argon for 6 hours. Copper triflate (0.009 g, 0.026 mmol) was added and the reaction continued as before overnight. The reaction was diluted with dichloromethane and water. The organic layer was separated and the aqueous layer was extracted with 3 portions of dichloromethane. The combined organics were washed with brine and dried over magnesium sulfate, filtered and concentrated in vacuo to afford a brown oil (147 mg). The residue was purified by mass directed auto-preparative chromatography to yield the formate salt of the title compound as a clear colourless oil (37 mg). This together with another portion of 8-[(3,3-difluoro-1-piperidinyl)methyl]-3-[(4-fluorophenyl)sulfonyl]quinoline (prepared in a similar manner to 8-[(3,3-difluoro-1-piperidinyl)methyl]-3-[(4-methylphenyl)sulfonyl]quinoline formate (4 mg)) was taken up in methanol (2 ml) was treated with 1.0M hydrochloric acid in diethyl ether (0.108 ml, 0.108 mmol) and then evaporated in vacuo to yield the title compound as an off-white solid (48 mg).

LC/MS [MH⁺] 421 consistent with molecular formula C₂₁H₁₉F₃N₂O₂S.

### EXAMPLE 39

### 8-[(3,3-Difluoro-1-piperidinyl)methyl]-3-[(4-methylphenyl)sulfonyl]quinoline hydrochloride

### E39

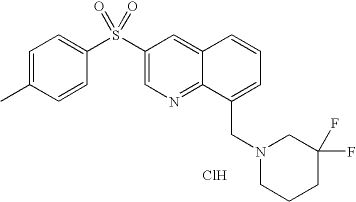

8-[(3,3-Difluoro-1-piperidinyl)methyl]-3-iodoquinoline (0.1 g, 0.26 mmol), sodium 4-methylphenylsulfinate hydrate (0.092 g, 0.52 mmol), potassium carbonate (36 mg, 0.26 mmol), copper (I) iodide (0.005 g, 0.03 mmol) and N,N-dimethylethylenediamine (5.59 μl, 0.052 mmol) were dissolved in dimethylsulfoxide (2 ml). The reaction mixture was heated at 100° C. for 2 hours. After cooling the reaction was diluted with dichloromethane and water. The organics were separated and the aqueous was extracted with 3 portions of dichloromethane. The combined organics were washed with brine, dried over magnesium sulfate, filtered and concentrated in vacuo to yield a yellow oil (132 mg). This was purified by mass directed auto-preparative chromatography to yield 8-[(3,3-difluoro-1-piperidinyl)methyl]-3-[(4-methylphenyl)sulfonyl]quinoline formate (75 mg). This was taken up in methanol (2 ml) and treated with 1.0M hydrochloric acid in diethyl ether (0.19 ml, 0.19 mmol) and then evaporated in vacuo to yield the title compound as an off-white solid (88 mg).

LC/MS [MH⁺] 417 consistent with molecular formula C₂₂H₂₂F₂N₂O₂S.

The following examples were prepared as hydrochloride salts by treatment of 8-[(3,3-difluoro-1-piperidinyl)methyl]-3-iodoquinoline (D20) with the appropriate sulfinate or sulfinic acid, as described for 8-[(3,3-Difluoro-1-piperidinyl)methyl]-3-[(4-methylphenyl)sulfonyl]quinoline hydrochloride (E39).

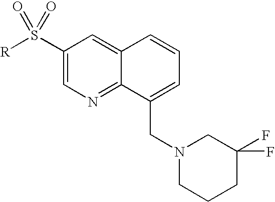

| | | | [MH]⁺ |
|---|---|---|---|
| Example | | | (mol. |
| No. | Chemical Name | R | Formula) |
| 40 | 3-[(4-Chlorophenyl)sulfonyl]-8-[(3,3-difluoro-1-piperidinyl)methyl]quinolinehydrochloride | 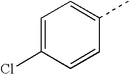 | 437C₂₁H₁₉³⁵ClF₂N₂O₂S |
| 41 | 3-[(4-Bromophenyl)sulfonyl]-8-[(3,3-difluoro-1-piperidinyl)methyl]quinolinehydrochloride | 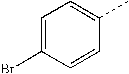 | 481C₂₁H₁₉⁷⁹BrF₂N₂O₂S |
| 42 | 3-{[3-Chloro-4-(methyloxy)phenyl]sulfonyl}-8-[(3,3-difluoro-1-piperidinyl)methyl]quinolinehydrochloride | 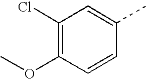 | 467C₂₂H₂₁³⁵ClF₂N₂O₃S |
| 43 | N-[5-({8-[(3,3-Difluoro-1-piperidinyl)methyl]-3-quinolinyl}sulfonyl)-2-(methyloxy)phenyl]acetamidehydrochloride | 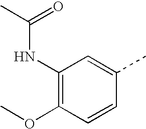 | 490C₂₄H₂₅F₂N₃O₄S |
| 44 | 3-{[3,4-Bis(methyloxy)phenyl]sulfonyl}-8-[(3,3-difluoro-1-piperidinyl)methyl]quinolinehydrochloride | 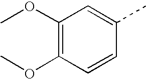 | 463C₂₃H₂₄F₂N₂O₄S |
| 45 | 3-[(3-Chloro-4-methylphenyl)sulfonyl]-8-[(3,3-difluoro-1-piperidinyl)methyl]quinolinehydrochloride | 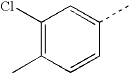 | 451C₂₂H₂₁³⁵ClF₂N₂O₂S |
| 46 | 8-[(3,3-Difluoro-1-piperidinyl)methyl]-3-(2-naphthalenylsulfonyl)quinolinehydrochloride | 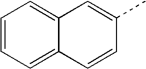 | 453C₂₅H₂₂F₂N₂O₂S |

Purification using mass directed auto-preparative chromatography was carried out using the following apparatus and conditions:

### Hardware

Waters 2525 Binary Gradient Module
Waters 515 Makeup Pump
Waters Pump Control Module
Waters 2767 Inject Collect
Waters Column Fluidics Manager
Waters 2996 Photodiode Array Detector
Waters ZQ Mass Spectrometer
Gilson 202 fraction collector
Gilson Aspec waste collector

### Software

Waters MassLynx version 4 SP2

### Column

Waters Atlantis, 30 mm×100 mm column packed with 5 μm stationary phase.

### Flow Rate

40 mls/min.

### Pharmacological Data

Compounds of the invention may be tested for in vitro biological activity at the 5HT₆ receptor in accordance with the following cyclase assay:

### Cyclase Assay

0.5 μl of test compound in 100% dimethylsulfoxide (DMSO) was added to a white, solid 384 well assay plate (for dose response measurements the top of the concentration range is 7.5 μM final). 10 μl of washed membranes of HeLa 5HT₆ cells (for preparation see WO 98/27081) in basic buffer (50 mM HEPES pH 7.4 (KOH), 1 mM MgCl₂, 100 mM NaCl, 1 μl/ml 3-isobutyl-1-methylxanthine (IBMX) (Sigma-Aldrich)) was added to all wells followed by 10 μl 2×ATP buffer (100 μl/ml ATP and 1 μl/ml 3-Isobutyl-1-methylxanthine (IBMX) (Sigma-Aldrich)) with 5-HT (at a concentration equivalent to a dose response of 4×EC₅₀). The resultant mixture was then incubated at room temperature for 30-45 minutes to allow cAMP production.

cAMP production was then measured using the DiscoveRx™ HitHunter™ chemiluminescence cAMP assay kit (DiscoveRx Corporation, 42501 Albrae Street, Fremont, Calif. 94538; Product Code: 90-0004L) or any other suitable cAMP measurement assay.

IC₅₀ values were estimated from arbitrary designated unit (ADU) measurements from a Perkin Elmer Viewlux instrument using a four parameter logistic curve fit within EXCEL (Bowen, W. P. and Jerman, J. C. (1995), Nonlinear regression using spreadsheets. Trends in Pharmacol. Sci., 16, 413-417). Functional Ki values were calculated using the method of Cheng, Y. C. and Prussof, W. H. (Biochemical Pharmacol (1973) 22 3099-3108). pIC50 and fpKi are the negative log 10 of the molar IC50 and functional Ki respectively.

The compounds of Examples E1-8, 10-17, 19-21, 23, 25-26, 28, 30 and 31 were tested in the above cyclase assay and showed antagonist potency for the 5-HT₆ receptor, having fpKi values >7.0 at human cloned 5-HT₆ receptors. The compounds of Examples E9, 18, 22, 24, 29 and 32 were also tested, having fpKi values ≧6.0 at human cloned 5-HT₆ receptors. The compound of Example E27 was also tested, having an fpKi value <6 at human cloned 5-HT₆ receptors. The compounds of Examples 29 and 33-46 were not tested in the above cyclase assay.

### Determination of Cannabinoid CB1 Receptor Agonist Activity

The cannabinoid CB1 receptor agonist activity of compounds of formula (I) was determined in accordance with the following experimental method.

### Experimental Method

Yeast (Saccharomyces cerevisiae) cells expressing the human cannabinoid CB1 receptor were generated by integration of an expression cassette into the ura3 chromosomal locus of yeast strain MMY23. This cassette consisted of DNA sequence encoding the human CB1 receptor flanked by the yeast GPD promoter to the 5′ end of CB1 and a yeast transcriptional terminator sequence to the 3′ end of CB1. MMY23 expresses a yeast/mammalian chimeric G-protein alpha subunit in which the C-terminal 5 amino acids of Gpa1 are replaced with the C-terminal 5 amino acids of human Gαi1/23 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD600/ml).

Agonists were prepared as 10 mM stocks in DMSO. EC50 values (the concentration required to produce 50% maximal response) were estimated using 4 fold dilutions dilutions of between 3- and 5-fold (BiomekFX, Beckman) into DMSO. Agonist solutions in DMSO (1% final assay volume) were transferred into black, clear bottom, microtitre plates from NUNC Greiner (96- or 384-well). Cells were suspended at a density of 0.2 OD600/ml in SC media lacking histidine, uracil, tryptophan, adenine and leucine and supplemented with 10 mM 3-aminotriazole, 0.1M sodium phosphate pH 7.0, and 120 μM fluorescein di-β-D-glucopyranoside (FDGlu). This mixture (50 ul per well for 384-well plates, 200 ul per well for 96-well plates) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a Spectrofluorfluorescence microtitre plate reader ((Tecan Spectrofluor or LJL Analyst excitation wavelength: 485 nm; emission wavelength: 535 nm). Tecan; excitation wavelength: 485 nm; emission wavelength: 535 nm). Fluorescence was plotted against compound concentration and iteratively curve fitted using a four parameter fit to generate a concentration effect value.

Efficacy (Emax) was calculated from the equation

Emax=Max[compound X]−Min[compound X]/Max[HU210]−Min[HU210]×100%
where Max [compound X] and Min [compound X] are the fitted maximum and minimum respectively from the concentration effect curve for compound X, and Max [HU210] and Min [HU210] are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC50[compound X]/EC50[HU210]
Where EC50[compound X] is the EC50 of compound X and EC50[HU210] is the EC50 of HU210.
pEC50 is the negative log of the EC50.
   Determination of cannabinoid CB2 Receptor Agonist Activity

The cannabinoid CB2 receptor agonist activity of compounds of formula (I) was determined in accordance with the following experimental method.

### Experimental Method

Yeast (Saccharomyces cerevisiae) cells expressing the human cannabinoid CB2 receptor were generated by integration of an expression cassette into the ura3 chromosomal locus of yeast strain MMY23. This cassette consisted of DNA sequence encoding the human CB2 receptor flanked by the yeast GPD promoter to the 5′ end of CB2 and a yeast transcriptional terminator sequence to the 3′ end of CB2. MMY23 expresses a yeast/mammalian chimeric G-protein alpha subunit in which the C-terminal 5 amino acids of Gpa1 are replaced with the C-terminal 5 amino acids of human Gαi1/23 (as described in Brown et al. (2000), Yeast 16:11-22). Cells were grown at 30° C. in liquid Synthetic Complete (SC) yeast media (Guthrie and Fink (1991), Methods in Enzymology, Vol. 194) lacking uracil, tryptophan, adenine and leucine to late logarithmic phase (approximately 6 OD₆₀₀/ml).

Agonists were prepared as 10 mM solutions in DMSO. EC₅₀ values (the concentration required to produce 50% maximal response) were estimated using 4 fold dilutions of between 3- and 5-fold (BiomekFX, Beckman) into DMSO. Agonist solutions in DMSO (1% final assay volume) were transferred into black microtitre plates from NUNC Greiner (384-well). Cells were suspended at a density of 0.2 OD₆₀₀/ml in SC media lacking histidine, uracil, tryptophan, adenine and leucine and supplemented with 10 mM 3-aminotriazole, 0.1M sodium phosphate pH 7.0, and 120 μM fluorescein di-β-D-glucopyranoside (FDGlu). This mixture (50 ul per well) was added to agonist in the assay plates (Multidrop 384, Labsystems). After incubation at 30° C. for 24 hours, fluorescence resulting from degradation of FDGlu to fluorescein due to exoglucanase, an endogenous yeast enzyme produced during agonist-stimulated cell growth, was determined using a fluorescence microtitre plate reader (Tecan Spectrofluor or LJL Analyst excitation wavelength: 485 nm; emission wavelength: 535 nm). Fluorescence was plotted against compound concentration and iteratively curve fitted using a four parameter fit to generate a concentration effect value.

Efficacy (Eₘₐₓ) was calculated from the equation

Eₘₐₓ=Max_{[compound X]}−Min_{[compound X]}/Max_{[HU210]}−Min_{[HU210]}×100%
where Max_{[compound X]} and Min_{[compound X]} are the fitted maximum and minimum respectively from the concentration effect curve for compound X, and Max_{[HU210]} and Min_{[HU210]} are the fitted maximum and minimum respectively from the concentration effect curve for (6aR,10aR)-3-(1,1′-Dimethylheptyl)-6a,7,10,10a-tetrahydro-1-hydroxy-6,6-dimethyl-6H-dibenzo[b,d]pyran-9-methanol (HU210; available from Tocris). Equieffective molar ratio (EMR) values were calculated from the equation

EMR=EC_{50[compound X]}/EC_{50[HU210]}
Where EC_{50 [compound X]} is the EC₅₀ of compound X and EC_{50 [HU210]} is the EC₅₀ of HU210.
pEC50 is the negative log of the EC50.

The compounds of Examples E1-9, 20, and 23-46 were tested for cannabinoid CB2 receptor agonist activity. The compounds of Examples E2, 4-5, 20, 25, 27-28, 30, 32-41 and 43-46 had pEC50 values >6 at the CB2 receptor. The compounds of Examples E1, 6-9, 23-24, 26, 29, 31 and 42 had pEC50 values ≧5 at the CB2 receptor. The compound of Example E3 had a pEC50 value <4.5 at the CB2 receptor. The compounds of Examples E10-19 and 21-22 were not tested for cannabinoid CB2 receptor agonist activity.

What is claimed is:

1-10. (canceled)

11. A compound of formula (I), or a pharmaceutically acceptable salt thereof: 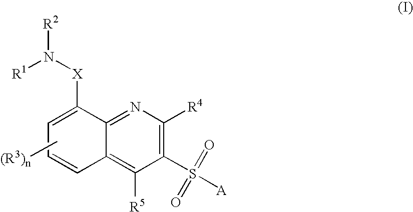
wherein:
R¹ and R² independently represent H, C₁₋₆ alkyl, or R¹ and R² together with the nitrogen atom to which they are attached form an optionally substituted 4 to 7 membered monocyclic heterocyclyl, a 9 to 11 membered bicyclic heterocyclyl, or a 10 membered spiro bicyclic heterocyclyl, any of which can optionally contain 1 or 2 further heteroatoms selected from O, N and S;
R³ represents halogen, —CN, —CF₃, —OCF₃, —OCHF₂, C₁₋₃ alkyl, C₁₋₃ alkoxy, —COC₁₋₃ alkyl, —NR⁶R⁷, or a group —CONR⁶R⁷;
R⁴ and R⁵ independently represent H, halogen, —CN, —CF₃, —OCF₃, —OCHF₂, C₁₋₃ alkyl, C₁₋₃ alkoxy, —COC₁₋₃ alkyl, —NR⁶R⁷, or a group —CONR⁶R⁷;
R⁶ and R⁷ independently represent H or C₁₋₃ alkyl;
X represents —(CH₂)ₘ— or —(CR⁸R⁹)—;
R⁸ and R⁹ independently represent H or C₁₋₃ alkyl;
m represents 2 to 4;
n represents 0 to 3; and
A represents an optionally substituted 6 to 10 membered aryl, an optionally substituted 5 to 7 membered monocyclic heteroaryl containing 1 to 3 heteroatoms selected from O, N and S, or a 9 to 10 membered bicyclic heteroaryl containing 1 to 3 heteroatoms selected from O, N and S.

12. A compound of claim 11, wherein R¹ and R² independently represent H, C₁₋₆ alkyl, or R¹ and R² together with the nitrogen atom to which they are attached form an optionally substituted 4 to 7 membered monocyclic heterocyclyl selected from the group consisting of azetidinyl, pyrrolidinyl, piperidinyl, piperazinyl, morpholinyl and thiomorpholinyl, or form an optionally substituted 1,4-dioxa-8-azaspiro[4.5]decane spiro bicyclic heterocyclyl.

13. A compound of claim 12, wherein the optional substituents of azetidinyl, pyrrolidinyl, piperidinyl, piperazinyl, morpholinyl, thiomorpholinyl and 1,4-dioxa-8-azaspiro[4.5]decane are selected from the group consisting of halogen, oxygen, C₁₋₄ alkyl, C₁₋₄ alkoxy, and —COC₁₋₄ alkyl.

14. A compound of claim 11, wherein A represents an optionally substituted phenyl or naphthyl.

15. A compound of claim 11 selected from the group consisting of
3-(Phenylsulfonyl)-8-(1-pyrrolidinylmethyl)quinoline hydrochloride (E1);
3-(Phenylsulfonyl)-8-(1-piperidinylmethyl)quinoline (E2);
8-[(4-Methyl-1-piperazinyl)methyl]-3-(phenylsulfonyl)quinoline (E3);
8-(4-(Morpholinylmethyl)-3-(phenylsulfonyl)quinoline (E4);
8-[(4,4-Difluoro-1-piperidinyl)methyl]-3-(phenylsulfonyl)quinoline (E5);
8-[(2,5-Dimethyl-1-pyrrolidinyl)methyl)-3-(phenylsulfonyl)quinoline (E6);
N,N-Dimethyl-1-[3-(phenylsulfonyl)quinolin-8-yl]methanamine (E7);
8-[(2-Methylpyrrolidin-1-yl)methyl]-3-(phenylsulfonyl)quinoline (E8);
N-Isopropyl-N-{[3-(phenylsulfonyl)quinolin-8-yl]methyl}propan-2-amine (E9);
3-(Phenylsulfonyl)-8-[2-(1-pyrrolidinyl)ethylquinoline hydrochloride (E10);
3-(Phenylsulfonyl)-8-[2-(1-piperidiny)ethyl]quinoline hydrochloride (E11);
3-(Phenylsulfonyl)-8-[3-(1-pyrrolidinyl)propyl]quinoline hydrochloride (E12);
3-(Phenylsulfonyl)-8-[3-(1-piperidinyl)propyl]quinoline (E13);
8-[3-(4-(Morpholinyl)propyl]-3-(phenylsulfonyl)quinoline (E14);
N,N-Dimethyl-3-[3-(phenylsulfonyl)-8-quinolinyl]-1-propanamine (E15);
3-(Phenylsulfonyl)-8-(1-pyrrolidin-1-ylethyl)quinoline hydrochloride (E16);
3-(Phenylsulfonyl)-8-(1-piperidin-1-ylethyl)quinoline (E17);
8-(1-Morpholin-4-ylethyl)-3-(phenylsulfonyl)quinoline (E18);
N,N-Dimethyl-1-[3-(phenylsulfonyl)quinolin-8-yl]ethanamine (E19);
N-Methyl-N-{1-[3-(phenylsulfonyl)quinolin-8-yl]ethyl}propan-2-amine hydrochloride (E20);
N-Ethyl-N-methyl-1-[3-(phenylsulfonyl)quinolin-8-yl]ethanamine hydrochloride (E21);
8-(1-Methyl-1-pyrrolidin-1-ylethyl)-3-(phenylsulfonyl)quinoline hydrochloride (E22);
3-[(4-Fluorophenyl)sulfonyl]-8-(1-pyrrolidinylmethyl)quinoline hydrochloride (E23);
8-[(4-Acetyl-1-piperazinyl)methyl]-3-(phenylsulfonyl)quinoline hydrochloride (E24);
N-{[3-(Phenylsulfonyl)-8-quinolinyl]methyl}-2-propanamine hydrochloride (E25);
N-Methyl-1-[3-(phenylsulfonyl)-8-quinolinyl]methanamine hydrochloride (E26);
8-[(3,3-Difluoropiperidin-1-yl)methyl]-3-(phenylsulfonyl)quinoline hydrochloride (E27);
8-[(4-Methoxypiperidin-1-yl)methyl]-3-(phenylsulfonyl)quinoline hydrochloride (E28);
8-[(2-Methyl-1-piperidinyl)methyl]-3-(phenylsulfonyl)quinoline hydrochloride (E29);
8-[(3-Methyl-1-piperidinyl)methyl]-3-(phenylsulfonyl)quinoline hydrochloride (E30);
8-(1,4-Dioxa-8-azaspiro[4.5]dec-8-ylmethyl)-3-(phenylsulfonyl)quinoline hydrochloride (E31);
8-[(1,1-Dioxido-4-thiomorpholinyl)methyl]-3-(phenylsulfonyl)quinoline hydrochloride (E32);
8-[(3,3-Difluoro-1-pyrrolidinyl)methyl]-3-(phenylsulfonyl)quinoline hydrochloride (E33);
8-[(3,3-Difluoro-1-azetidinyl)methyl]-3-(phenylsulfonyl)quinoline hydrochloride (E34);
8-[1-(3,3-Difluoro-1-azetidinyl)ethyl]-3-(phenylsulfonyl)quinoline (E35);
8-[1-(3,3-Difluoro-1-pyrrolidinyl)ethyl]-3-(phenylsulfonyl)quinoline (E36);
8-[1-(3,3-Difluoro-1-piperidinyl)ethyl]-3-(phenylsulfonyl)quinoline (E37);
8-[(3,3-Difluoro-1-piperidinyl)methyl]-3-[(4-fluorophenyl)sulfonyl]quinoline hydrochloride (E38);
8-[(3,3-Difluoro-1-piperidinyl)methyl]-3-[(4-methylphenyl)sulfonyl]quinoline hydrochloride (E39);
3-[(4-Chlorophenyl)sulfonyl]-8-[(3,3-difluoro-1-piperidinyl)methyl]quinoline hydrochloride (E40);
3-[(4-Bromophenyl)sulfonyl]-8-[(3,3-difluoro-1-piperidinyl)methyl]quinoline hydrochloride (E41);
3-{[3-Chloro-4-(methyloxy)phenyl]sulfonyl}-8-[(3,3-difluoro-1-piperidinyl)methyl]quinoline hydrochloride (E42);
N-[5-({8-[(3,3-Difluoro-1-piperidinyl)methyl]-3-quinolinyl}sulfonyl)-2-(methyloxy)phenyl]acetamide hydrochloride (E43);
3-{[3,4-Bis(methyloxy)phenyl]sulfonyl}-8-[(3,3-difluoro-1-piperidinyl)methyl]quinoline hydrochloride (E44);
3-[(3-Chloro-4-methylphenyl)sulfonyl]-8-[(3,3-difluoro-1-piperidinyl)methyl]quinoline hydrochloride (E45); and
8-[(3,3-Difluoro-1-piperidinyl)methyl]-3-(2-naphthalenylsulfonyl)quinoline hydrochloride (E46).

16. A pharmaceutical composition which comprises a compound claim 11 and a pharmaceutically acceptable carrier or excipient.

17. A method of treating a mammal suffering from a condition selected from the group consisting of anxiety, depression, obsessive compulsive disorders, cognitive memory disorders, Parkinsons Disease, sleep disorders, feeding disorders, panic attacks, withdrawal from drug abuse, schizophrenia, stroke, disorders associated with spinal trauma, head injury, or both, said method comprising administering to said mammal a therapeutically effective amount of a compound of claim 11.

18. The method of claim 17 wherein said mammal is a human.

19. A method of treating a mammal suffering from a condition selected from the group consisting of cognitive disorders associated with Alzheimers disease, age related cognitive decline, mild cognitive impairment, vascular dementia, Parkinson's disease, schizophrenia, IBS, and obesity, said method comprising administering to said mammal a therapeutically effective amount of a compound of claim 11 or a pharmaceutically acceptable salt thereof.

20. The method of claim 19 wherein said mammal is a human.
